(19) United States
(12) Patent Application Publication
(10) Pub. No.: US 2008/0026933 A1
(43) Pub. Date: Jan. 31, 2008

(54) 1,1'-Diphosphinoferrocenes Having 2,2'-Bound Achiral Or Chiral Radicals

(76) Inventors: Benoit Pugin, Munchenstein (CH); Xiang Dong Feng, Qingdao (CN); Felix Spindler, Starrkirch-wil (CH)

Correspondence Address: WENDEROTH, LIND & PONACK, L.L.P., 2033 K STREET N. W., SUITE 800, WASHINGTON, DC, 20006-1021, US

(21) Appl. No.: 11/631,608

(22) PCT Filed: Jul. 4, 2005
(86) PCT No.: PCT/EP05/53171

§ 371 Date: Jan. 5, 2007

(30) Foreign Application Priority Data
Jul. 5, 2004 (CH) 1126/04
Oct. 26, 2004 (CH) 1773/04
Nov. 10, 2004 (CH) 1853/04

(51) Int. Cl. C07F 15/02; B01J 31/00; C07D 207/08

(52) U.S. Cl. 502/102; 548/402; 556/14; 556/22

(57) ABSTRACT

In ferrocene diphosphines of formula (I), in which R represents, for example, methyl or phenyl, the catalytic properties of corresponding metal complexes can, in many instances, be distinctly influenced by structural changes on one or both of the CP rings, and the catalytic reaction with regard to selected substrates can be optimized and significantly improved. Diphosphine ligands of this type are accessible by using novel production methods.

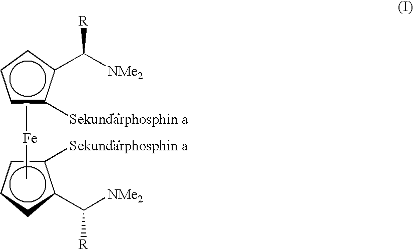

The present invention relates to 2,2′-diphosphinoferrocenes which have a radical having a chiral α carbon atom or an achiral or chiral radical bound via a CH₂ group bound in the 1,1′ positions and contain at least one further substituent in the cyclopentadienyl rings; processes for preparing them; metal complexes of transition metals with these diphosphines as ligands; and the use of the metal complexes as homogeneous catalysts in asymmetric or symmetric addition reactions and also a process for the preferably asymmetric hydrogenation of prochiral unsaturated organic compounds.

Chiral diphosphines have proven to be valuable ligands in transition metal complexes which are used as homogeneous catalysts for asymmetric addition reactions and in particular hydrogenations. A large number of chiral ligands of the diphosphine type are known. It remains an unsolved problem in the field of this stereoselective catalysis that it is not possible to predict which ligands will enable good catalyst activity and stereoselectivity to be achieved in a particular reaction with a defined substrate. For this reason, suitable ligands are nowadays identified by trials. When a suitable ligand has been found, it is very advantageous to be able to carry out optimization in respect of its structure and properties for the target reaction.

Ferrocenediphosphines of the mandyphos (trivial name) type 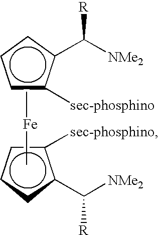
where R is, for example, methyl or phenyl, have been known for a relatively long time and are described, inter alia, in a summary fashion by P. Knochel et al. in Tetrahedron: Asymmetry 10 (1999), pages 375 to 384. Metal complexes of such ligands can, in the case of particular substrates, lead to better hydrogenation results than complexes with other diphosphine ligands. The properties of these ligands can be varied only by the choice of the substituents R and/or the substituents in the secondary phosphino groups. It would be extremely desirable to broaden the range of use of the ligands by utilizing further optimization possibilities by means of structural changes on one or both cyclopentadienyl rings. However, no such structural modifications nor methods of achieving them have become known.

It has now surprisingly been found that introduction of substituents and/or variation of the secondary amino group in one or both cyclopentadienyl rings of the type of ligand mentioned at the outset can in many cases significantly influence the catalytic properties of corresponding metal complexes and enable catalytic reactions to be better optimized and significantly improved for selected substrates. It has also been found that such novel diphosphine ligands can be obtained via novel preparative processes and can be prepared in a modular fashion via defined intermediates.

The present invention firstly provides compounds of the formula I or I′ in the form of racemates, mixtures of stereoisomers or optically pure stereoisomers, 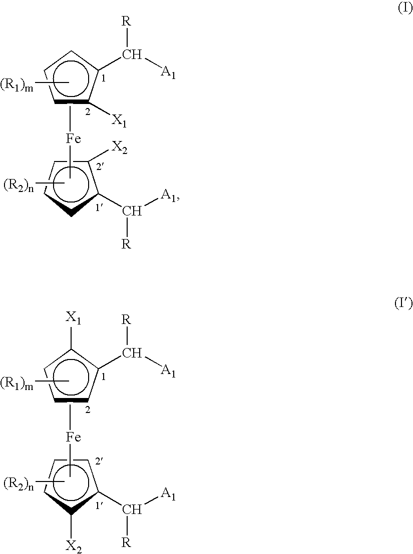
where
R is hydrogen or unsubstituted or F—, Cl—, OH—, C₁-C₄-alkyl- or C₁-C₄-alkoxy-substituted C₁-C₈-alkyl, C₃-C₈-cycloalkyl, C₆-C₁₀-aryl or C₇-C₁₁-aralkyl;
X₁ and X₂ are each, independently of one another, a secondary phosphino group;
A₁ is an amino group; or
A₁ is an —OR₃ radical, where R₃ is hydrogen or unsubstituted or F—, C₁-C₄-alkyl-, C₁-C₄-alkoxy-, phenyl- or N(C₁-C₄-alkyl)₂-substituted C₁-C₁₈-alkyl, C₃-C₈-cycloalkyl, C₆-C₁₀-aryl, C₇-C₁₁-aralkyl or C₁-C₁₈-acyl;
R₁ and R₂ are each, independently of one another, a halogen atom or a substituent bound to the cyclopentadienyl rings via a C atom, N atom, S atom, Si atom, a P(O) group or P(S) group;
m is from 1 to 3, and
n is 0 or from 1 to 3.

Among the stereoisomers, those having an R,S,R′,S′, R,R,R′,R′, S,R,S′,R and S,S,S′,S′ configuration and mixtures thereof are preferred.

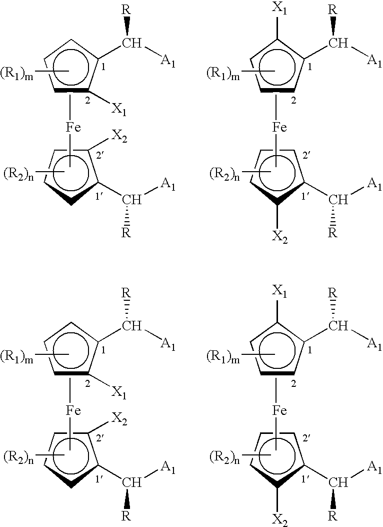

A C₁-C₈-alkyl radical R can be linear or branched and an alkyl radical R₁ is preferably C₁-C₄-alkyl. These can be, for example, methyl, ethyl, n- or i-propyl and n-, i- or t-butyl and also the isomers of pentyl, hexyl, heptyl and octyl. Examples of substituted alkyl are fluoromethyl, difluoromethyl, trifluoromethyl, trifluoroethyl, hydroxymethyl, β-hydroxyethyl, methoxymethyl, ethoxymethyl and β-methoxyethyl. The alkyl radical is preferably linear. An alkyl radical R₁ is preferably methyl or ethyl.

A cycloalkyl radical R is preferably C₅-C₈-cycloalkyl. It can be, for example, cyclopropyl, cyclobutyl, cyclopentyl, cyclohexyl, cycloheptyl or cyclooctyl, which may, for example, be substituted by F, C₁-C₄-alkyl or C₁-C₄-alkoxy. Preferred cycloalkyl radicals are cyclopentyl and cyclohexyl.

A C₆-C₁₀-aryl radical R can be, for example, phenyl or naphthyl. An aryl radical R₁ is preferably phenyl, which may be unsubstituted or substituted by F, Cl, C₁-C₄-alkyl or C₁-C₄-alkoxy.

An aralkyl radical R is preferably phenyl-C₁-C₄-alkyl and particularly preferably benzyl or β-phenylethyl, with the phenyl group being able to be substituted by F, Cl, C₁-C₄-alkyl or C₁-C₄-alkoxy.

In a preferred embodiment, R in the compounds of the formula I is hydrogen, methyl, ethyl, cyclohexyl, benzyl or phenyl

The secondary phosphino groups X₁ and X₂ can be two identical or two different hydrocarbon radicals. The secondary phosphino groups X₁ and X₂ preferably each contain two identical hydrocarbon radicals. Furthermore, the secondary phosphino groups X₁ and X₂ can be identical or different. The secondary phosphino groups X₁ and X₂ are preferably identical.

The hydrocarbon radicals can be unsubstituted or substituted and/or contain heteroatoms selected from the group consisting of O, S, and N. They can contain from 1 to 22, preferably from 1 to 18 and particularly preferably from 1 to 14, carbon atoms. A preferred sec-phosphino group contains two identical or different radicals selected from the group consisting of linear or branched C₁-C₁₂-alkyl; unsubstituted or C₁-C₆-alkyl- or C₁-C₆-alkoxy-substituted C₅-C₁₂-cycloalkyl or C₅-C₁₂-cycloalkyl-CH₂—; phenyl, naphthyl, furyl or benzyl; or halogen- (for example F—, Cl— or Br—), C₁-C₆-alkyl-, C₁-C₆-haloalkyl- (for example trifluoromethyl-), C₁-C₆-alkoxy-, C₁-C₆-haloalkoxy- (for example trifluoromethoxy-), (C₆H₅)₃Si—, (C₁-C₁₂-alkyl)₃Si—, sec-amino- or —CO₂—C₁-C₆-alkyl (for example —CO₂CH₃)-substituted phenyl and benzyl.

Examples of alkyl substituents on P, which preferably contain from 1 to 6 carbon atoms, are methyl, ethyl, n-propyl, i-propyl, n-butyl, i-butyl, t-butyl and the isomers of pentyl and hexyl. Examples of unsubstituted or alkyl-substituted cycloalkyl substituents on P are cyclopentyl, cyclohexyl, methylcyclopentyl, ethylcyclopentyl, dimethylcyclopentyl, methylcyclohexyl and ethylcyclohexyl and dimethylcyclohexyl. Examples of alkyl-, alkoxy-, haloalkyl-, haloalkoxy- and halogen-substituted phenyl and benzyl substituents on P are o-, m- or p-fluorophenyl, o-, m- or p-chlorophenyl, difluorophenyl or dichlorophenyl, pentafluorophenyl, methylphenyl, dimethylphenyl, trimethylphenyl, ethylphenyl, methylbenzyl, methoxyphenyl, dimethoxyphenyl, trifluoromethylphenyl, bistrifluoromethylphenyl, tristrifluoromethylphenyl, trifluoromethoxyphenyl, bistrifluoromethoxyphenyl and 3,5-dimethyl-4-methoxyphenyl.

Preferred secondary phosphino groups are those which contain identical radicals selected from the group consisting of C₁-C₆-alkyl, unsubstituted cyclopentyl or cyclohexyl or cyclopentyl or cyclohexyl bearing from 1 to 3 C₁-C₄-alkyl or C₁-C₄-alkoxy groups as substituents, benzyl and in particular phenyl which are unsubstituted or substituted by from 1 to 3 C₁-C₄-alkyl, C₁-C₄-alkoxy, F, Cl, C₁-C₄-fluoroalkyl or C₁-C₄-fluoroalkoxy substituents. The substituent F can also be present four or five times.

The sec-phosphino group preferably corresponds to the formula —PR₃R₄, where R₃ and R₄ are each, independently of one another, a hydrocarbon radical which has from 1 to 18 carbon atoms and is unsubstituted or substituted by halogen, C₁-C₆-alkyl, C₁-C₆-haloalkyl, C₁-C₆-alkoxy, C₁-C₆-haloalkoxy, (C₁-C₄-alkyl)₂amino, (C₆H₅)₃Si, (C₁-C₁₂-alkyl)₃Si or —CO₂—C₁-C₆-alkyl and/or contains heteroatoms O.

R₃ and R₄ are preferably identical radicals selected from the group consisting of linear or branched C₁-C₆-alkyl, unsubstituted cyclopentyl or cyclohexyl or cyclopentyl or cyclohexyl bearing from 1 to 3 C₁-C₄-alkyl or C₁-C₄-alkoxy groups as substituents, furyl, norbornyl, adamantyl, unsubstituted benzyl or benzyl bearing from 1 to 3 C₁-C₄-alkyl or C₁-C₄-alkoxy groups as substituents and in particular unsubstituted phenyl or phenyl substituted by from 1 to 3 C₁-C₄-alkyl, C₁-C₄-alkoxy, —NH₂, —N(C₁-C₆-alkyl)₂, OH, F, Cl, C₁-C₄-fluoroalkyl or C₁-C₄-fluoroalkoxy substituents.

R₃ and R₄ are particularly preferably identical radicals selected from the group consisting of C₁-C₆-alkyl, cyclopentyl, cyclohexyl, furyl and unsubstituted phenyl or phenyl substituted by from 1 to 3 C₁-C₄-alkyl, C₁-C₄-alkoxy and/or C₁-C₄-fluoroalkyl groups.

The secondary phosphino groups X₁ and X₂ can be cyclic sec-phosphino groups, for example those of the formulae 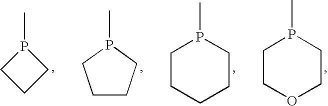
which are unsubstituted or monosubstituted or multiply substituted by —OH, C₁-C₈-alkyl, C₄-C₈-cycloalkyl, C₁-C₆-alkoxy, C₁-C₄-alkoxy-C₁-C₄-alkyl, phenyl, C₁-C₄-alkyl or C₁-C₄-alkoxy-phenyl, benzyl, C₁-C₄-alkylbenzyl or C₁-C₄-alkoxybenzyl, benzyloxy, C₁-C₄-alkylbenzyloxy or C₁-C₄-alkoxybenzyloxy or C₁-C₄-alkylidenedioxyl.

The substituents can be bound to the P atom in one or both a positions in order to introduce chiral carbon atoms. The substituents in one or both a positions are preferably C₁-C₄-alkyl or benzyl, for example methyl, ethyl, n- or i-propyl, benzyl or —CH₂—O—C₁-C₄-alkyl or —CH₂—O—C₆-C₁₀-aryl.

Substituents in the β,γ positions can, for example, be C₁-C₄-alkyl, C₁-C₄-alkoxy, benzyloxy or —O—CH₂—O—, —O—CH(C₁-C₄-alkyl)-O—, —O—C(C₁-C₄-alkyl)₂-O— and —O—CH(C₆-C₁₀-aryl)-O—. Some examples are methyl, ethyl, methoxy, ethoxy, —O—CH(phenyl)-O—, —O—CH(methyl)-O— and —O—C(methyl)₂-O—.

An aliphatic 5- or 6-membered ring or benzene can be fused onto two adjacent carbon atoms in the radicals of the above formulae.

Other known secondary phosphino radicals which are suitable are those of cyclic and chiral phospholanes having seven carbon atoms in the ring, for example those of the formulae 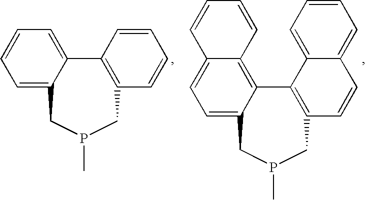
in which the aromatic rings may be substituted by C₁-C₄-alkyl, C₁-C₄-alkoxy, C₁-C₄-alkoxy-C₁-C₂-alkyl, phenyl, benzyl, benzyloxy or C₁-C₄-alkylidenedioxyl or C₁-C₄-alkylenedioxyl (cf. US 2003/0073868 A1 and WO 02/048161).

Depending on the type of substitution and the number of substituents, the cyclic phosphino radicals can be C-chiral, P-chiral or C- and P-chiral.

The cyclic sec-phosphino group can, for example, correspond to one of the formulae (only one of the possible diastereomers is shown), 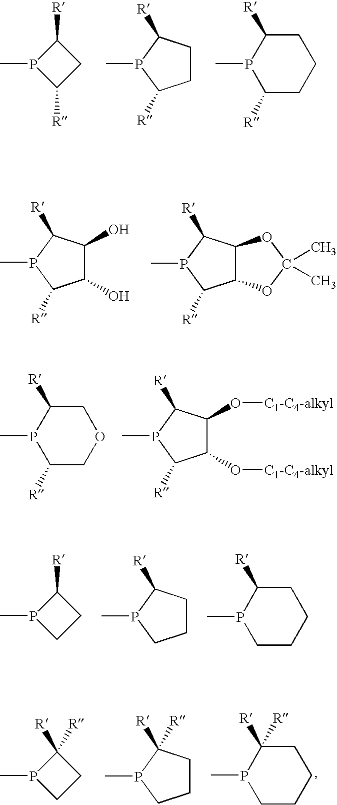
where
the radicals R′ and R″ are each C₁-C₄-alkyl, for example methyl, ethyl, n- or i-propyl, benzyl or —CH₂—O—C₁-C₄-alkyl or —CH₂—O—C₆-C₁₀-aryl and R′ and R″ are identical or different. When R′ and R″ are bound to the same carbon atom, they can together also be C₄-C₅-alkylene.

In preferred embodiments, the groups X₁ and X₂ in the compounds of the formulae I and I′ are preferably identical or different acyclic sec-phosphino groups in each case selected from the group consisting of —P(C₁-C₆-alkyl)₂, —P(C₅-C₈-cycloalkyl)₂, —P(C₇-C₁₂-bicycloalkyl)₂, —P(o-furyl)₂, —P(C₆H₅)₂, —P[2-(C₁-C₆-alkyl)C₆H₄]₂, —P[3-(C₁-C₆-alkyl)C₆H₄]₂, —P[4-(C₁-C₆-alkyl)C₆H₄]₂, —P[2-(C₁-C₆-alkoxy)C₆H₄]₂, —P[3-(C₁-C₆-alkoxy)C₆H₄]₂, —P[4-(C₁-C₆alkoxy)C₆H₄]₂, —P[2-(trifluoromethyl)C₆H₄]₂, —P[3-(trifluoromethyl)C₆H₄]₂,
—P[4-(trifluoromethyl)C₆H₄]₂, —P[3,5-bis(trifluoromethyl)C₆H₃]₂, —P[3,5-bis(C₁-C₆-alkyl)₂C₆H₃]₂, —P[3,5-bis(C₁-C₆-alkoxy)₂C₆H₃]₂, —P[3,4,5-tris(C₁-C₆-alkoxy)₂C₆H₃]₂ and —P[3,5-bis(C₁-C₆-alkyl)₂₋₄-(C₁-C₆-alkoxy)C₆H₂]₂, or cyclic phosphino groups selected from the group consisting of 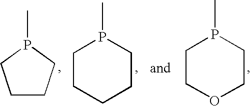
which are unsubstituted or monosubstituted or multiply substituted by C₁-C₄-alkyl, C₁-C₄-alkoxy, C₁-C₄-alkoxy-C₁-C₂-alkyl, phenyl, benzyl, benzyloxy, C₁-C₄-alkylidenedioxyl or unsubstituted or phenyl-substituted methylenedioxyl.

Some specific examples are —P(CH₃)₂, —P(i-C₃H₇)₂, —P(n-C₄H₉)₂, —P(i-C₄H₉)₂, —P(C₆H₁₁)₂, —P(norbornyl)₂, —P(o-furyl)₂, —P(C₆H₅)₂, P[2-(methyl)C₆H₄]₂, P[3-(methyl)C₆H₄]₂, —P[4-(methyl)C₆H₄]₂, —P[2-(methoxy)C₆H₄]₂, —P[3-(methoxy)C₆H₄]₂, —P[4-(methoxy)C₆H₄]₂, —P[3-(trifluoromethyl)C₆H₄]₂, —P[4-(trifluoromethyl)C₆H₄]₂, —P[3,5-bis(trifluoromethyl)C₆H₃]₂, —P[3,5-bis(methyl)C₆H₃]₂, —P[3,5-bis(methoxy)C₆H₃]₂, —P[3,4,5-tri(methoxy)C₆H₂]₂ and —P[3,5-bis(methyl)₂₋₄-(methoxy)C₆H₂]₂ and groups of the formulae 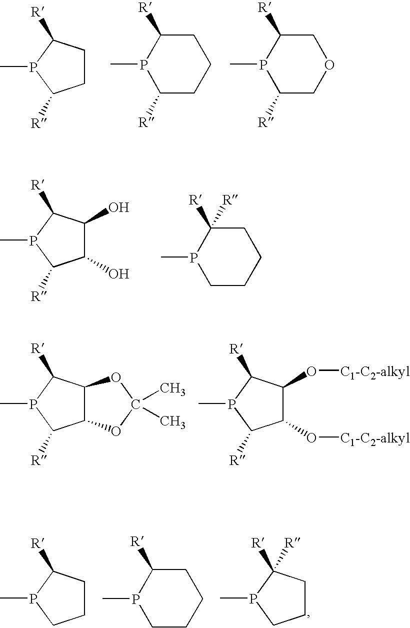
where
R′ is methyl, ethyl, methoxy, ethoxy, phenoxy, benzyloxy, methoxymethyl, ethoxymethyl or benzyloxymethyl and R″ has the same meanings as R′.

The amino group A₁ can be —NH₂, —NHR₅ or —NR₅R₆, where R₅ and R₆ are each, independently of one another, a substituted or unsubstituted aliphatic, cycloaliphatic or aromatic hydrocarbon radical or R₅ and R₆ together with the N atom form an N-heterocyclic ring which may contain further heteroatoms from the group consisting of O, S or N(C₁-C₄-alkyl). The N-heterocyclic ring preferably has from 3 to 12, more preferably from 3 to 8 and particularly preferably from 5 to 8, ring members. The groups —NHR₅ or —NR₅R₆ preferably contain a total of from 2 to 24 carbon atoms, more preferably from 2 to 16 carbon atoms and particularly preferably from 2 to 12 carbon atoms.

The hydrocarbon radicals and N-heterocyclic rings can be monosubstituted or poly-substituted, for example monosubstituted to trisubstituted, preferably monosubstituted or disubstituted, by, for example, halogen (F or Cl, in particular F), —CN, —NR₀₁R₀₂, —C(O)—O—R₀₃, —C(O)—NR₀₃R₀₄, —O—(O)C—R₀₄, —R₀₁N—(O)C—R₀₄, C₁-C₄-alkyl, C₁-C₄-alkoxy, C₁-C₄-alkoxy-C₁-C₄-alkyl, C₁-C₄-alkylthio-C₁-C₄-alkyl, C₅-C₆-cycloalkyl, C₅-C₆-cycloalkoxy, phenyl, benzyl, phenoxy or benzyloxy, where R₀₁ and R₀₂ are each, independently of one another, hydrogen, C₁-C₄-alkyl, cyclopentyl, cyclohexyl, phenyl, benzyl or R₀₁ and R₀₂ together are tetramethylene, pentamethylene or 3-oxapentane-1,5-diyl, R₀₃ is hydrogen, C₁-C₈-alkyl, C₅-C₆-cycloalkyl, phenyl or benzyl and R₀₄ is C₁-C₁₈-alkyl and preferably C₁-C₁₂-alkyl, C₁-C₄-haloalkyl, C₁-C₄-hydroxyalkyl, C₅-C₈-cycloalkyl (for example cyclopentyl, cyclohexyl), C₆-C₁₀-aryl (for example phenyl or naphthyl) or C₇-C₁₂-aralkyl (for example benzyl).

An amino group A₁ can correspond to the formula —NHR₅ and R₅R₆N—, where R₅ and R₆ are each, independently of one another, substituted or unsubstituted C₁-C₁₂-alkyl and preferably C₁-C₆-alkyl, C₃-C₈-cycloalkyl and preferably C₅-C₆-cycloalkyl, C₆-C₁₀-aryl and preferably phenyl and C₇-C₁₁-aralkyl and preferably benzyl, with any substitution being as described above, or R₅ and R₆ together with the N atom form a 3- to 8-membered and preferably 5- to 8-membered N-heterocyclic ring which may be unsubstituted or substituted as described above.

Examples of alkyl, which is preferably linear, are methyl, ethyl, propyl, butyl, pentyl, hexyl, heptyl and octyl. Examples of cycloalkyl are cyclopentyl, cyclohexyl and cyclooctyl. Examples of cycloalkyl are, in particular, cyclopentyl and cyclohexyl. R₅ and R₆ together are preferably tetramethylene, pentamethylene, 3-oxapentylene or 3-(C₁-C₄-alkyl)N-pentylene when the sec-amino forms an N-heterocyclic ring.

When the radicals R₅ and R₆ contain asymmetric carbon atoms, these are located, for example, in the γ position and preferably the α or β positions relative to the N atom. Preferred substituents for forming asymmetric carbon atoms are C₁-C₄-alkyl, C₅-C₆-cycloalkyl, phenyl, benzyl, C₁-C₄-alkoxy, C₁-C₄-alkoxymethyl, C₁-C₄-alkoxyethyl, (C₁-C₄-alkyl)₂N—, (C₁-C₄-alkyl)₂N-methyl and (C₁-C₄-alkyl)₂N-ethyl.

In a preferred embodiment, R₅ and R₆ are each methyl, ethyl, the isomers of propyl and butyl, phenyl, benzyl, cyclohexyl or R₅ and R₆ together are tetramethylene, pentamethylene or 3-oxapentylene, which may be unsubstituted or substituted by C₁-C₄-alkyl, C₅-C₆-cycloalkyl, phenyl, benzyl, C₁-C₄-alkoxy, C₁-C₄-alkoxymethyl, C₁-C₄-alkoxyethyl, (C₁-C₄-alkyl)₂N—, (C₁-C₄-alkyl)₂N-methyl and (C₁-C₄-alkyl)₂N-ethyl.

An alkyl radical R₃ can be C₁-C₁₂-alkyl and preferably C₁-C₆-alkyl. A cycloalkyl radical R₃ can be C₅-C₆-cycloalkyl. An aryl radical R₃ is preferably phenyl and an aralkyl radical R₃ is preferably benzyl. An acyl radical R₃ preferably contains from 1 to 12 and particularly preferably from 1 to 8 carbon atoms. The acyl is preferably derived from a carboxylic acid, for example formic acid, acetic acid, propionic acid, butyric acid, chloroacetic acid, hydroxyacetic acid, methoxyacetic acid or benzoic acid.

In a preferred embodiment of the compounds of the formula I, the substituents R₁ and R₂ are present once (m is 1 and n is 0), each present once (m and n are each 1), present twice (either m or n is 2) or present three times (m is 2 and n is 1) on the cyclopentadienyl ring or rings. Preferred positions for the substituents R₁ and R₂ are the 3, 3′, 5 and 5′ positions. Preferred substitution patterns are the 3 position, the 3 and 3′ positions, the 5 position and the 5 and 5′ positions. The sum m+n is preferably from 1 to 5, more preferably from 1 to 4 and particularly preferably from 1 to 3.

Substituents R₁ and R₂ may in turn be monosubstituted or polysubstituted, for example monosubstituted to trisubstituted, preferably monosubstituted or disubstituted, by, for example, halogen (F, Cl or Br, in particular F), —OH, —SH, —CH(O), —CN, —NR₀₁R₀₂, —C(O)—O—R₀₃, —S(O)—O—R₀₃, —S(O)₂—O—R₀₃, —P(OR₀₃)₂, —P(O)(OR₀₃)₂, —C(O)—NR₀₁R₀₂, —S(O)—NR₀₁R₀₂, —S(O)₂—NR₀₁R₀₂, —O—(O)C—R₀₄, —R₀₁N—(O)C—R₀₄, —R₀₁N—S(O)—R₀₄, —R₀₁N—S(O)₂—R₀₄, C₁-C₄-alkyl, C₁-C₄-alkoxy, C₁-C₄-alkylthio, C₅-C₆-cycloalkyl, phenyl, benzyl, phenoxy or benzyloxy, where R₀₁ and R₀₂ are each, independently of one another, hydrogen, C₁-C₄-alkyl, cyclopentyl, cyclohexyl, phenyl, benzyl or R₀₁ and R₀₂ together are tetramethylene, pentamethylene or 3-oxapentane-1,5-diyl, R₀₃ is hydrogen, C₁-C₈-alkyl, C₅-C₆-cycloalkyl, phenyl or benzyl and R₀₄ is C₁-C₁₈-alkyl and preferably C₁-C₁₂-alkyl, C₁-C₄-haloalkyl, C₁-C₄-hydroxyalkyl, C₅-C₈-cycloalkyl (for example cyclopentyl, cyclohexyl), C₆-C₁₀-aryl (for example phenyl or naphthyl) or C₇-C₁₂-aralkyl (for example benzyl).

The substituted or unsubstituted substituents R₁ and R₂ can, for example, be C₁-C₁₂-alkyl, preferably C₁-C₈-alkyl and particularly preferably C₁-C₄-alkyl. Examples are methyl, ethyl, n- or i-propyl, n-, i- or t-butyl, pentyl, hexyl, heptyl, octyl, decyl and dodecyl.

The substituted or unsubstituted substituents R₁ and R₂ can, for example, be C₅-C₈-cycloalkyl, preferably C₅-C₆-cycloalkyl. Examples are cyclopentyl, cyclohexyl and cyclooctyl.

The substituted or unsubstituted substituents R₁ and R₂ can, for example, be C₅-C₈-cycloalkyl-alkyl, preferably C₅-C₆-cycloalkylalkyl. Examples are cyclopentylmethyl, cyclohexylmethyl or cyclohexylethyl and cyclooctylmethyl.

The substituted or unsubstituted substituents R₁ and R₂ can, for example, be C₆-C₁₈-aryl and preferably C₆-C₁₀-aryl. Examples are phenyl and naphthyl.

The substituted or unsubstituted substituents R₁ and R₂ can, for example, be C₇-C₁₂-aralkyl (for example benzyl or 1-phenyleth-2-yl).

The substituted or unsubstituted substituents R₁ and R₂ can, for example, be tri(C₁-C₄-alkyl)Si or triphenylsilyl. Examples of trialkylsilyl are trimethylsilyl, triethylsilyl, tri-n-propylsilyl, tri-n-butylsilyl and dimethyl-t-butylsilyl.

The substituents R₁ and R₂ can, for example, be halogen. Examples are F, Cl and Br.

The substituted or unsubstituted substituents R₁ and R₂ can, for example, be thio radicals or sulfoxide or sulfone radicals of the formulae —SR₀₅, —S(O)R₀₅ and —S(O)₂R₀₅, where R₀₅ is C₁-C₁₂-alkyl, preferably C₁-C₈-alkyl and particularly preferably C₁-C₄-alkyl; C₅-C₈-cycloalkyl, preferably C₅-C₆-cycloalkyl; C₆-C₁₈-aryl and preferably C₆-C₁₀-aryl; or C₇-C₁₂-aralkyl. Examples of these hydrocarbon radicals have been mentioned above.

The substituents R₁ and R₂ can, for example, be —CH(O), —C(O)—C₁-C₄-alkyl or —C(O)—C₆-C₁₀-aryl.

The substituted or unsubstituted substituents R₁ and R₂ can, for example, be —CO₂R₀₃ or —C(O)—NR₀₁R₀₂ radicals, where R₀₁, R₀₂ and R₀₃ have the meanings given above, including the preferences.

The substituted or unsubstituted substituents R₁ and R₂ can, for example, be —S(O)—O—R₀₃, —S(O)₂—O—R₀₃, —S(O)—NR₀₁R₀₂ and —S(O)₂—NR₀₁R₀₂ radicals, where R₀₁, R₀₂ and R₀₃ have the meanings given above, including the preferences.

The substituted or unsubstituted substituents R₁ and R₂ can, for example, be —P(OR₀₃)₂ or —P(O)(OR₀₃)₂ radicals, where R₀₃ has the meanings given above, including the preferences.

The substituted or unsubstituted substituents R₁ and R₂ can, for example, be —P(O)(R₀₃)₂ or —P(S)(OR₀₃)₂ radicals, where R₀₃ has the meanings given above, including the preferences.

In a preferred group of substituents R₁ and R₂, these are selected from among C₁-C₄-alkyl, substituted or unsubstituted phenyl, tri(C₁-C₄-alkyl)Si, triphenylsilyl, halogen (in particular F, Cl and Br), —SR₀₆, —CH₂OH, —CHR₀₆OH, —CR₀₆R′₀₆OH, —CH₂O—R₀₆, —CH(O), —CO₂H, —CO₂R₀₆, where R₀₆ is a hydrocarbon radical having from 1 to 10 carbon atoms, and —P(O)(R₀₃)₂, where R₀₃ has the meanings given above. R₁ and R₂ are particularly preferably C₁-C₄-alkyl, in particular methyl, and tri(C₁-C₄-alkyl)Si, in particular trimethylsilyl.

Examples of substituted or unsubstituted substituents R₁ and R₂ are methyl, ethyl, n- and i-propyl, n-, i- and t-butyl, pentyl, hexyl, cyclohexyl, cyclohexylmethyl, phenyl, benzyl, trimethylsilyl, F, Cl, Br, methylthio, methylsulfonyl, methylsulfoxyl, phenylthio, phenylsulfonyl, phenylsulfoxyl, —CH(O), —C(O)OH, —C(O)—OCH₃, —C(O)—OC₂H₅, —C(O)—NH₂, —C(O)—NHCH₃, —C(O)—N(CH₃)₂, —SO₃H, —S(O)—OCH₃, —S(O)—OC₂H₅, —S(O)₂—OCH₃, —S(O)₂—OC₂H₅, —S(O)—NH₂, —S(O)NHCH₃, —S(O)—N(CH₃)₂, —S(O)—NH₂, —S(O)₂—NHCH₃, —S(O)₂—N(CH₃)₂, —P(OH)₂, —PO(OH)₂, —P(OCH₃)₂, —P(OC₂H₅)₂, —PO(OCH₃)₂, —PO(OC₂H₅)₂, trifluoromethyl, methylcyclohexyl, methylcyclohexylmethyl, methylphenyl, dimethylphenyl, methoxyphenyl, dimethoxyphenyl, hydroxymethyl, β-hydroxyethyl, γ-hydroxypropyl, —CH₂NH₂, —CH₂N(CH₃)₂, —CH₂CH₂NH₂, —CH₂CH₂N(CH₃)₂, methoxymethyl, ethoxymethyl, methoxyethyl, ethoxyethyl, HS—CH₂—, HS—CH₂CH₂—, CH₃S—CH₂—, CH₃S—CH₂CH₂—, —CH₂—C(O)OH, —CH₂CH₂—C(O)OH, —CH₂—C(O)OCH₃, —CH₂CH₂—C(O)OCH₃, —CH₂—C(O)NH₂, —CH₂CH₂—C(O)NH₂, —CH₂—C(O)—N(CH₃)₂, —CH₂CH₂—C(O)N(CH₃)₂, —CH₂—SO₃H, —CH₂CH₂—SO₃H, —CH₂—SO₃CH₃, —CH₂CH₂—SO₃CH₃, —CH₂—SO₂NH₂, —CH₂—SO₂N(CH₃)₂, —CH₂—PO₃H₂, —CH₂CH₂—PO₃H₂, —CH₂—PO(OCH₃), —CH₂CH₂—PO(OCH₃)₂, —C₆H₄—C(O)OH, —C₆H₄—C(O)OCH₃, —C₆H₄—S(O)₂OH, —C₆H₄—S(O)₂OCH₃, —CH₂—O—C(O)CH₃, —CH₂CH₂—O—C(O)CH₃, —CH₂—NH—C(O)CH₃, —CH₂CH₂—NH—C(O)CH₃, —CH₂—O—S(O)₂CH₃, —CH₂CH₂—O—S(O)₂CH₃, —CH₂—NH—S(O)₂CH₃, —CH₂CH₂—NH—S(O)₂ CH₃, —P(O)(C₁-C₈-alkyl)₂, —P(S)(C₁-C₈-alkyl)₂, —P(O)(C₆-C₁₀-aryl)₂, —P(S)(C₆-C₁₀-aryl)₂, —C(O)—C₁-C₈-alkyl and —C(O)—C₆-C₁₀-aryl.

The compounds of the formula I can be prepared by various methods, depending on the positions in which substituents are to be introduced. The ortho positions in the cyclopentadienyl (hereinafter referred to as cp for short) relative to the groups X₁ and X₂ are the 3 or 3′ positions. The ortho positions in the cp relative to the A₁CHR— groups are the 5 or 5′ positions. The 4 positions are located between the 3 and 5 positions.

If only one substituent is to be introduced in the 3 position of a cp ring, the preparation can start out from known and sometimes commercially available 1,1′-bis(1-sec-aminoeth-1-yl)-ferrocenes which are, in a process step a), metallated by means of metallation reagents such as alkyllithium and the metal is subsequently replaced by bromine. The ortho position relative to the bromine can then again be lithiated selectively by means of Li amides in a process step b) and desired substituents can subsequently be introduced by reaction with appropriate electrophiles in a process step c). In the final reaction step d), the bromine atoms in the 2,2′ positions are firstly metallated (for example by means of alkyllithium) and subsequently reacted with X₁-halide to introduce the secondary phosphino groups.

The metallations of ferrocenes as in the first process step are known reactions which are described, for example, by T. Hayashi et al., Bull. Chem. Soc. Jpn. 53 (1980), pages 1138 to 1151 or in Jonathan Clayden Organolithiums: Selectivity for Synthesis (Tetrahedron Organic Chemistry Series), Pergamon Press (2002). The alkyl in the alkyllithium can, for example, contain from 1 to 4 carbon atoms. Methyllithium and butyllithium are frequently used. Magnesium Grignard compounds are preferably compounds of the formula (C₁-C₄-alkyl)MgX₀, where X₀ is Cl, Br or I.

The reaction is advantageously carried out at low temperatures, for example from 20 to −100° C., preferably from 0 to −80° C. The reaction time is from about 2 to 20 hours. The reaction is advantageously carried out under an inert protective gas, for example nitrogen or noble gases such as argon.

The reaction is advantageously carried out in the presence of inert solvents. Such solvents can be used alone or as a combination of at least two solvents. Examples of solvents are aliphatic, cycloaliphatic and aromatic hydrocarbons and also open-chain or cyclic ethers.

Specific examples are petroleum ether, pentane, hexane, cyclohexane, methylcyclohexane, benzene, toluene, xylene, diethyl ether, dibutyl ether, tert-butyl methyl ether, ethylene glycol dimethyl or diethyl ether, tetrahydrofuran and dioxane.

The halogenation in process step a) is generally carried out directly after the metallation in the same reaction mixture, with similar reaction conditions as in the metallation being maintained. Preference is given to using from 1 to 1.4 equivalents of a halogenating reagent. Halogenating reagents are, for example, halogens (Cl₂, Br₂, I₂), interhalogens (Cl—Br, Cl—I) and aliphatic, perhalogenated hydrocarbons (Cl₃C—CCl₃ or BrF₂C—CF₂Br) for introduction of Cl, Br or I; or N-fluorobis(phenyl)sulfonylamine for introduction of fluorine.

The metallation in process step a) and the halogenation proceed regioselectively and the intermediates are obtained in high yields. The reaction is also stereoselective because of the presence of the chiral group A₁CHR—. Furthermore, should this be necessary, optical isomers can also be separated at this stage, for example by chromatography using chiral columns.

In process step b), the ferrocene skeleton is once again metallated regioselectively in the ortho position relative to the halogen atom in the same cyclopentadienyl ring, with metal amides being sufficient to replace the acidic H atom in the ortho position relative to the halogen atom. Use is made of at least from 1 to 5 equivalents of an aliphatic Li sec-amide or a Cl-, Br- or IMG-sec-amide per CH group in the cyclopentadienyl ring of the ferrocene.

Aliphatic Li sec-amide or halogen-Mg-sec-amide can be derived from sec-amines which contain from 2 to 18, preferably from 2 to 12 and particularly preferably 2 to 10, carbon atoms. The aliphatic radicals bound to the N atom can be alkyl, cycloalkyl or cycloalkylalkyl, or the N atom together with the aliphatic radicals can form N-heterocyclic rings having from 4 to 12 and preferably from 5 to 7 carbon atoms. Examples of radicals bound to the N atom are methyl, ethyl, n- and i-propyl, n-butyl, pentyl, hexyl, cyclopentyl, cyclohexyl and cyclohexylmethyl. Examples of N-heterocyclic rings are pyrrolidine, piperidine, morpholine, N-methylpiperazine, 2,2,6,6-tetramethylpiperidine, and azanorbornane. In a preferred embodiment, the amides correspond to the formulae Li—N(C₃-C₄-alkyl)₂ or X₂Mg—N(C₃-C₄-alkyl)₂, where alkyl is, in particular, i-propyl. In another preferred embodiment, the amides correspond to Li(2,2,6,6-tetramethylpiperidine).

In process step c), radicals of electrophilic compounds are introduced with replacement of the metal (M). It is possible to use from 1 to 1.2 equivalents of reactive electrophilic compound per reacting ═CM group in an aromatic compound. However, a significant excess of up to 2.5 equivalents can also be used.

The reaction is advantageously carried out at low temperatures, for example from 20 to −100° C., preferably from 0 to −80° C. The reaction is advantageously carried out under an inert protective gas, for example noble gases such as argon or else nitrogen. After addition of the reactive electrophilic compound, the reaction mixture is advantageously allowed to warm to room temperature or is heated to elevated temperatures, for example up to 100° C. and preferably up to 50° C., and stirred for some time under these conditions to complete the reaction.

The reaction is advantageously carried out in the presence of inert solvents. Such solvents can be used alone or as a combination of at least two solvents. Examples of solvents are aliphatic, cycloaliphatic and aromatic hydrocarbons and also open-chain or cyclic ethers. Specific examples are petroleum ether, pentane, hexane, heptane, cyclohexane, methylcyclohexane, benzene, toluene, xylene, diethyl ether, dibutyl ether, tert-butyl methyl ether, ethylene glycol dimethyl or diethyl ether, tetrahydrofuran and dioxane.

Examples of reactive electrophilic compounds for forming radicals R₁ and R₂ are:

halogens (Cl₂, Br₂, I₂), interhalogens (Cl—Br, Cl—I) and aliphatic, perhalogenated hydrocarbons (Cl₃C—CCl₃ or BrF₂C—CF₂Br, N-fluorobis(phenyl)sulfonylamine) for introduction of F, Cl, Br or I;

CO₂ for introduction of the carboxyl group —CO₂H;

chlorocarbonates or bromocarbonates [Cl—C(O)—OR] for introduction of a carboxylate group, where R is a hydrocarbon radical (alkyl, cycloalkyl, cycloalkylalkyl, aryl, aralkyl, heteroaryl, heteroaralkyl) which has from 1 to 18, preferably from 1 to 12 and particularly preferably from 1 to 8, carbon atoms and is substituted by inert substituents such as sec-phosphino di(C₁-C₈-alkyl)₂N—, —C(O)—OC₁-C₈-alkyl, or —OC₁-C₈-alkyl (reactive groups such as Cl, Br or I are also included under inert substituents if groups such as —CHO which are more reactive toward a metal or a metal group are at the same time present in compounds of the formula I or if Cl and Br, Cl and I or Br and I are simultaneously bound to a preferably aromatic hydrocarbon radical);

di(C₁-C₄-alkyl)formamides, for example dimethylformamide or diethylformamide, for introduction of the —CH(O) group;

di(C₁-C₄-alkyl)carboxamides for introduction of a —C(O)—R group;

aldehydes which may be unsubstituted or substituted by sec-phosphino in the group R for introduction of a —CH(OH)—R group or paraformaldehyde for introduction of the —CH₂OH group;

symmetrical or unsymmetrical ketones which may be unsubstituted or substituted by sec-phosphino in the group R or Rₐ for introduction of a —C(OH)RRₐ group, where Rₐ independently has one of the meanings of R, or R and Rₐ together form a cycloaliphatic ring having from 3 to 8 ring members;

epoxides for introduction of a —C—C—OH group in which the carbon atoms may be substituted by H or R;

an Eschenmoser salt of the formula (CH₃)₂N⁺═CH₂xl;

imines R—CH═N—Rₐ for introduction of the —CH(R)—NH—Rₐ group, where Rₐ independently has one of the meanings of R, or R and Rₐ together form a cycloaliphatic ring having from 3 to 8 ring members; R and Rₐ are not simultaneously hydrogen;

imines R—C(R_{b})═N—Rₐ for introduction of the —C(R)(R_{b})—NH—Rₐ group, where Rₐ independently has one of the meanings of R, or R and R′ together form a cycloaliphatic ring having from 3 to 8 ring members, R_{b} independently has one of the meanings of R, or R and R_{b} together form a cycloaliphatic ring having from 3 to 8 ring members;

hydrocarbon monohalides and heterohydrocarbon monohalides, in particular chlorides, bromides and iodides, for introduction of hydrocarbon and heterohydrocarbon radicals (for example C₁-C₁₈-alkyl, C₆-C₁₄-aryl, C₇-C₁₄-aralkyl);

halohydrocarbons and haloheterohydrocarbons having halogen atoms of differing reactivity, in particular combinations of chlorine with bromine or iodine, bromine with iodine or two bromine or iodine atoms, for introduction of hydrocarbon and heterohydrocarbon radicals (for example C₁-C₁₈-alkyl, C₆-C₁₄-aryl, C₇-C₁₄-aralkyl);

alkenyl halides, in particular chlorides, bromides and iodides, for introduction of alkenyl groups such as allyl or vinyl;

tri(C₁-C₈-alkyl)silyl halides (chlorides, bromides) for introduction of the tri(C₁-C₈-alkyl)-Si— group, triphenylsilyl halides for introduction of the triphenylsilyl group;

phosphoric ester monohalides (chlorides, bromides) for introduction of phosphonic ester groups such as (CH₃O)₂(O)P—, (C₂H₅O)(O)P—, (cyclohexylO)₂(O)P—, (ethylenedioxyl)(O)P—;

thiophosphoric ester monohalides (chlorides, bromides) for introduction of thiophosphonic ester groups such as (CH₃O)₂(S)P—, (C₂H₅O)(S)P—, (cyclohexylO)₂(S)P—, (ethylenedioxyl)(S)P—;

organic disulfides R—SS—R for introduction of the —SR group; and

sulfur (S₈) for introduction of the —SH group.

In the processes described below, only one of the possible stereoisomers is shown as structural formula. A person skilled in the art will know the other stereoisomers.

To introduce substituents in the 3 and 3′ positions of the cp rings, it is possible to start out from known and sometimes commercially available mandyphos ligands which are, for example,
a) reacted with organic peroxides to form the corresponding phosphine oxides, preferably of the formula II 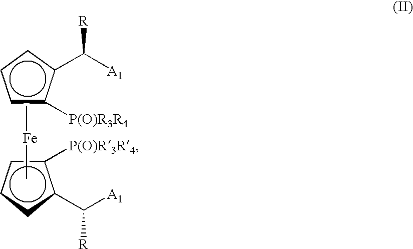
where R, R₃ and R₄ have the meanings given above and R₁₃ and R₁₄ independently have one of the meanings of R₃ and R₄ and A₁ is sec-amino,
b) the compounds of the formula II are preferably lithiated and then reacted with an electrophile to introduce the radicals R₁ and R₂ in the 3 and 3′ positions and form compounds of the formula III, 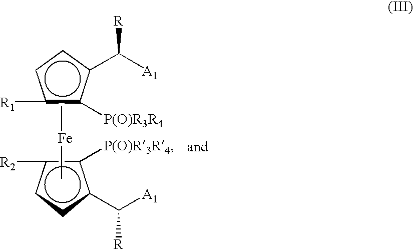
c) the compounds of the formula III are reduced to form compounds of the formula I. The substituents introduced have to be inert under the reduction conditions.

The oxidation in process step a) is advantageously carried out in solvents (such as those mentioned above) and at temperatures of from about −30 to 50° C. The reaction conditions in process step b) are analogous to the conditions described above. The reduction in process step c) can be effected catalytically or by means of chemical hydriding reagents, for example metal hydrides [Li(AlH₄)], alkylboranes or alkoxyboranes, alkylsilanes or alkoxysilanes or alkylstannanes or alkoxystannanes, if appropriate together with Lewis acids such as metal alkoxides (titanium tetraalkoxides). The reaction is advantageously carried out in the presence of solvents and at temperatures of from 0 to 150° C., depending on the reactivity of the hydriding reagent.

Substitutions in the 5 and 5′ positions proceed particularly well when R in compounds of the formula I is hydrogen or a substituent and an O- or N-containing substituent which directs the metal in the metallation in the 5 and 5′ positions is additionally present in the radical A₁.

The invention provides a process for preparing compounds of the formula Ia 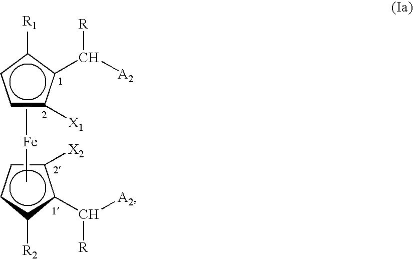
where
R, X₁, X₂, R₁ and R₂ have the meanings given above, with R₂ being hydrogen when R is not hydrogen, A₂ is an open-chain or cyclic sec-amino group having at least one asymmetric carbon atom when R is hydrogen or A₂ is an open-chain or cyclic, achiral or chiral sec-amino group substituted by di(C₁-C₄-alkyl)amino or C₁-C₄-alkoxy when R is not hydrogen, which is characterized in that a compound of the formula IV 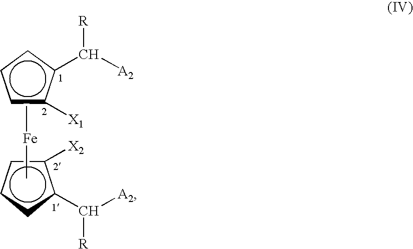
is metallated by means of one or at least two equivalents of metallation reagent, preferably alkyllithium, either only in the 5 position or in the 5 and 5′ positions and then reacted with electrophiles to introduce the groups R₁ and R₂.

The compounds of the formula IV are firstly valuable intermediates for the process of the invention and secondly valuable ligands for homogeneous catalysts of the transition metals. The compounds of the formula IV in which R is hydrogen or unsubstituted or F—, Cl—, OH—, C₁-C₄-alkyl- or C₁-C₄-alkoxy-substituted C₁-C₈-alkyl, C₃-C₈-cycloalkyl, C₆-C₁₀-aryl or C₇-C₁₁-aralkyl; A₂ is open-chain or cyclic sec-amino having at least one asymmetric carbon atom are also provided by the invention.

The process conditions have been described above and will be illustrated in the examples. Since the metallation in step a) can be carried out stepwise when R is hydrogen, not only monosubstituted and disubstituted compounds but also compounds having different substituents can be prepared by this process.

An open-chain or cyclic sec-amino group A₂ can correspond to the formula R₅R₆N—, where R₅ and R₆ are each, independently of one another, C₁-C₁₂-alkyl and preferably C₁-C₆-alkyl, C₃-C₈-cycloalkyl and preferably C₅-C₆-cycloalkyl or together with the N atom form a 3- to 8-membered and preferably 5- to 8-membered N-heterocyclic ring and at least one of R₅ and R₆ and the heterocyclic group may contain an O- or N-containing substituent.

Examples of alkyl, which is preferably linear, are methyl, ethyl, propyl, butyl, pentyl, hexyl, heptyl and octyl. Examples of cycloalkyl are cyclopentyl, cyclohexyl and cyclooctyl. Examples of cycloalkyl are, in particular, cyclopentyl and cyclohexyl. R₅ and R₆ together are preferably tetramethylene, pentamethylene, 3-oxapentylene or 3-(C₁-C₄-alkyl)N-pentylene when the sec-amino group forms an N-heterocyclic ring. Suitable substituents are, for example, C₁-C₄-alkoxy, C₁-C₄-alkoxymethyl, C₁-C₄-alkoxyethyl, (C₁-C₄-alkyl)₂N—, (C₁-C₄-alkyl)₂N-methyl and (C₁-C₄-alkyl)₂N-ethyl. The substituents are, for example, located in the γ position and preferably the α or β positions relative to the N atom of the sec-amino group. R₅ and R₆ can also be substituted by C₁-C₄-alkyl, C₅-C₆-cycloalkyl, phenyl or benzyl.

In a preferred embodiment, R₅ and R₆ are each methyl, ethyl, cyclohexyl or R₅ and R₆ together are tetramethylene, pentamethylene or 3-oxapentylene which are substituted by C₁-C₄-alkoxy, C₁-C₄-alkoxymethyl, C₁-C₄-alkoxyethyl, (C₁-C₄-alkyl)₂N—, (C₁-C₄-alkyl)₂N-methyl and (C₁-C₄-alkyl)₂N-ethyl and, if desired, additionally by C₁-C₄-alkyl, C₅-C₆-cycloalkyl, phenyl or benzyl.

Particularly preferred examples are those of the formulae 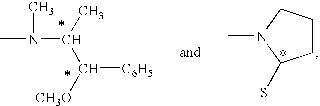
where S is C₁-C₄-alkoxy, C₁-C₄-alkoxymethyl, C₁-C₄-alkoxyethyl, (C₁-C₄-alkyl)₂N—, (C₁-C₄-alkyl)₂N-methyl or (C₁-C₄-alkyl)₂N-ethyl.

Compounds of the formula IV can be obtained in a simple manner from compounds of the formula V, 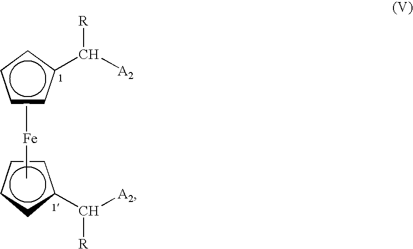
by metallating these and then reacting them with sec-phosphine halides. Compounds of the formula V can be obtained by substitution of corresponding acetoxy or amine compounds or their salts with amines A₂H.

Diastereomers of the formula Ib which are different from the diastereomers obtained by the above process 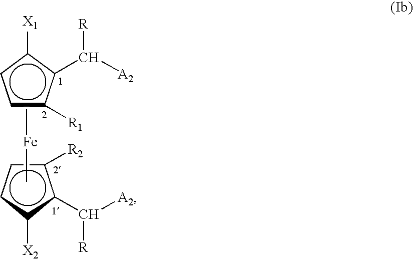
can be prepared by means of an alteration of the reaction sequence in which a metallated (lithiated) compound of the formula V is firstly reacted with an electrophile to introduce the radicals R₁ and R₂, then metallated (lithiated) again and then reacted with sec-phosphine halides.

Compounds of the formula I in which the 3,3′,5,5′ positions are substituted can likewise be obtained from compounds of the formula V by
a) metallating (lithiating) these and then halogenating them to form compounds of the formula VI, 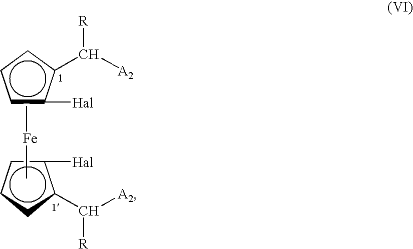
where Hal is Cl, Br or I, preferably Br,
b) metallating the compounds of the formula VI by means of a secondary lithium amide and then reacting them with an electrophile to introduce the radicals R₁ and R₂ and form compounds of the formula VII, 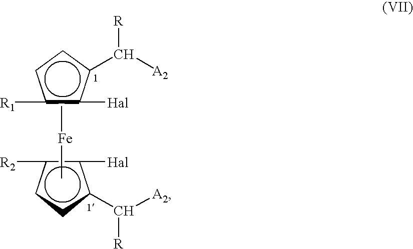
c) metallating (lithiating) the compounds of the formula VII and then reacting them with sec-phosphine halides to form compounds of the formula VIII, 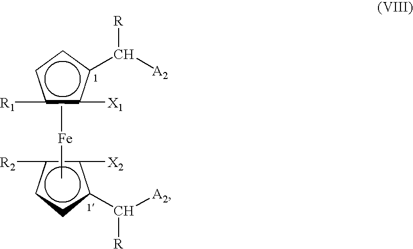
metallating (lithiating) the compounds of the formula VIII and then reacting them with an electrophile to introduce the radicals R₁ and R₂ and form compounds of the formula IX, 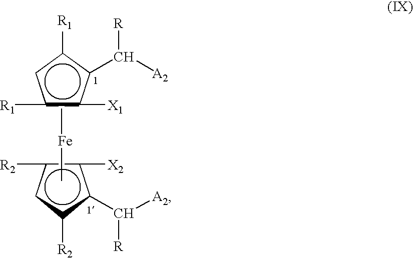
where the two radicals R₁ and the two radicals R₂ can be identical or different radicals.

Compounds of the formula I which are substituted in the 4 and 4′ positions can be obtained from compounds of the formula IV by
a) metallating (lithiating) these and then halogenating them to form compounds of the formula X (Hal is Cl, Br or I), preferably brominating them (Hal is Br), 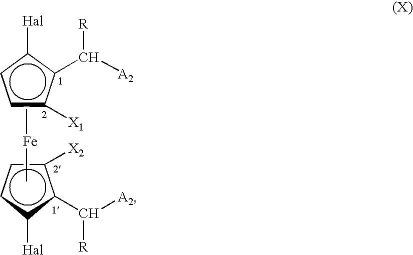
b) lithiating the compounds of the formula X by means of a secondary lithium amide and then reacting them with an electrophile to introduce the radicals R₁ and R₂ and form compounds of the formula XI, 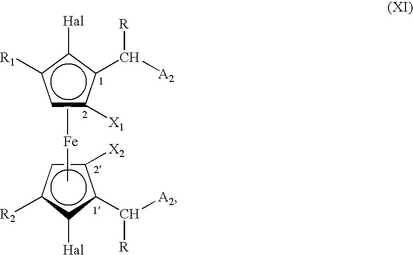
c) and, if desired, replacing the halogen atoms in the compounds of the formula XI by lithium by reaction with alkyllithium in order then to replace Hal by hydrogen by hydrolytic cleavage with water or react them with an electrophile to introduce the radicals R₁ and R₂, where the two radicals R₁ and the two radicals R₂ can be identical or different radicals.

The secondary amino group A₁ in the novel compounds of the formula I (A₁ can also have the same meaning as A₂) can be modified further by, for example, replacing the group A₁ by acetoxy in a known manner using acetic anhydride. The acetoxy group can be hydrolyzed to form a hydroxyl group or replaced by reaction with any desired alcohols R₃OH or amines. The hydroxyl compounds can also be esterified or etherified. Such processes are described in the literature, cf., for example, T. Hayashi et al., Bull. Chem. Soc. Jpn. 53 (1980), pages 1138 to 1151.

The metal complexes of the invention are homogeneous catalysts or catalyst precursors which can be activated under the reaction conditions which can be used for asymmetric addition reactions on prochiral, unsaturated, organic compounds, cf. E. Jacobsen, A. Pfaltz, H. Yamamoto (Eds.), Comprehensive Asymmetric Catalysis I to III, Springer Verlag, Berlin, 1999, and B. Cornils et al., in Applied Homogeneous Catalysis with Organometallic Compounds, Volume 1, Second Edition, Wiley VCH-Verlag (2002).

The novel compounds of the formulae I, I′ and IV are ligands for complexes of metals selected from the group of the TM8 metals, in particular from the group consisting of Ru, Rh and Ir, excellent catalysts or catalyst precursors for asymmetric syntheses, for example the asymmetric hydrogenation of prochiral, unsaturated, organic compounds. If prochiral unsaturated organic compounds are used, a very high excess of optical isomers can be induced in the synthesis of organic compounds and a high chemical conversion can be achieved in short reaction times. The enantioselectivities and catalyst activities which can be achieved are excellent and in an asymmetric hydrogenation are considerably higher than when using the known “Kagan ligands” mentioned at the outset. Furthermore, such ligands can also be used in other asymmetric addition or cyclization reactions.

The invention further provides complexes of metals selected from the group of TM8 metals with one of the compounds of the formula I or I′ or IV as ligand.

Possible metals are, for example, Cu, Ag, Au, Ni, Co, Rh, Pd, Ir, Ru and Pt. Preferred metals are rhodium and iridium and also ruthenium, platinum and palladium.

Particularly preferred metals are ruthenium, rhodium and iridium.

The metal complexes can, depending on the oxidation number and coordination number of the metal atom, contain further ligands and/or anions. They can also be cationic metal complexes. Such analogous metal complexes and their preparation are widely described in the literature.

The metal complexes can, for example, correspond to the general formulae XII and XIII,
A₃MeLᵣ (XII),
(A₃MeLᵣ)^{(z+)}(E⁻)_{z} (XIII),
where A₃ is one of the compounds of the formula I or I′ or IV,
L represents identical or different monodentate, anionic or nonionic ligands or L represents identical or different bidentate, anionic or nonionic ligands;
r is 2, 3 or 4 when L is a monodentate ligand or n is 1 or 2 when L is a bidentate ligand;
z is 1, 2 or 3;
Me is a metal selected from the group consisting of Rh, Ir and Ru; with the metal having the oxidation states 0, 1, 2, 3 or 4;
E⁻ is the anion of an oxo acid or complex acid; and
the anionic ligands balance the charge of the oxidation states 1, 2, 3 or 4 of the metal.

The preferences and embodiments described above apply to the compounds of the formulae I and 1′.

Monodentate nonionic ligands can, for example, be selected from the group consisting of olefins (for example ethylene, propylene), solvating solvents (nitriles, linear or cyclic ethers, unalkylated or N-alkylated amides and lactams, amines, phosphines, alcohols, carboxylic esters, sulfonic esters), nitrogen monoxide and carbon monoxide.

Suitable polydentate anionic ligands are, for example, allyls (allyl, 2-methallyl) or deprotonated 1,3-diketo compounds such as acetylacetonate and also cyclopentadienyl.

Monodentate anionic ligands can, for example, be selected from the group consisting of halide (F, Cl, Br, I), pseudohalide (cyanide, cyanate, isocyanate) and anions of carboxylic acids, sulfonic acids and phosphonic acids (carbonate, formate, acetate, propionate, methylsulfonate, trifluoro methylsulfonate, phenylsulfonate, tosylate).

Bidentate nonionic ligands can, for example, be selected from the group consisting of linear or cyclic diolefins (for example hexadiene, cyclooctadiene, norbornadiene), dinitriles (malononitrile), unalkylated or N-alkylated carboxylic diamides, diamines, diphosphines, diols, dicarboxylic diesters and disulfonic diesters.

Bidentate anionic ligands can, for example, be selected from the group consisting of the anions of dicarboxylic acids, disulfonic acids and diphosphonic acids (for example of oxalic acid, malonic acid, succinic acid, maleic acid, methylenedisulfonic acid and methylene-diphosphonic acid).

Preferred metal complexes also include those in which E⁻ is —Cl⁻, —Br⁻, —I⁻, ClO₄⁻, CF₃SO₃⁻, CH₃SO₃⁻, HSO₄⁻, (CF₃SO₂)₂N⁻, (CF₃SO₂)₃C⁻, tetraarylborates such as B(phenyl)₄⁻, B[3,5-bis(trifluoromethyl)phenyl]₄⁻, B[3,5-dimethylphenyl]₄⁻, B(C₆F₅)₄⁻ and B(4-methylphenyl)₄⁻, BF₄⁻, PF₆⁻, SbCl₆⁻, AsF₆⁻ or SbF₆⁻.

Particularly preferred metal complexes which are particularly suitable for hydrogenations correspond to the formulae XIV and XV,
[A₃Me₂Y₁Z] (XIV),
[A₃Me₂Y₁]⁺E₁⁻ (XV),
where
A₃ is one of the compounds of the formula I or I′ or IV;
Me₂ is rhodium or iridium;
Y₁ represents two olefins or one diene;
Z is Cl, Br or I; and
E₁⁻ is the anion of an oxo acid or complex acid.

The embodiments and preferences described above apply to the compounds of the formulae I and I′.

An olefin Y₁ can be a C₂-C₁₂-, preferably C₂-C₆- and particularly preferably C₂-C₄-olefin. Examples are propene, 1-butene and in particular ethylene. The diene can contain from 5 to 12 and preferably from 5 to 8 carbon atoms and can be an open-chain, cyclic or polycyclic diene. The two olefin groups of the diene are preferably connected by one or two CH₂ groups. Examples are 1,4-pentadiene, cyclopentadiene, 1,5-hexadiene, 1,4-cyclohexadiene, 1,4- or 1,5-heptadiene, 1,4- or 1,5-cycloheptadiene, 1,4- or 1,5-octadiene, 1,4- or 1,5-cyclooctadiene and norbornadiene. Y preferably represents two ethylene molecules or 1,5-hexadiene, 1,5-cyclooctadiene or norbornadiene.

In formula IX, Z is preferably Cl or Br. Examples of E₁ are BF₄⁻; ClO₄⁻, CF₃SO₃⁻, CH₃SO₃⁻, HSO₄⁻, B(phenyl)₄⁻, B[3,5-bis(trifluoromethyl)phenyl]₄⁻, PF₆⁻, SbCl₆⁻, AsF₆⁻ or SbF₆⁻.

The metal complexes of the invention are prepared by methods known in the literature (cf. U.S. Pat. No. 5,371,256, U.S. Pat. No. 5,446,844, U.S. Pat. No. 5,583,241 and E. Jacobsen, A. Pfaltz, H. Yamamoto (Eds.), Comprehensive Asymmetric Catalysis I to II, Springer Verlag, Berlin, 1999, and references cited therein).

The ruthenium complexes can, for example, correspond to the formula XVI,
[RuₐH_{b}Z_{c}(A₃)_{d}Lₑ]_{f}(E^{k})_{g}(S)ₕ (XVI),
where
Z is Cl, Br or I; A₃ is a compound of the formula I or I′ or IV; L represents identical or different ligands; E⁻ is the anion of an oxo acid, mineral acid or complex acid; S is a solvent capable of coordination as ligand; and a is from 1 to 3, b is from 0 to 4, c is from 0 to 6, d is from 1 to 3, e is from 0 to 4, f is from 1 to 3, g is from 1 to 4, h is from 0 to 6 and k is from 1 to 4, with the overall complex being uncharged.

The above-described preferences for Z, A₃, L and E⁻ apply to the compounds of the formula VIII. The ligands L can also be arenes or heteroarenes (for example benzene, naphthalene, methylbenzene, xylene, cumene, 1,3,5-mesitylene, pyridine, biphenyl, pyrrole, benzimidazole or cyclopentadienyl) and metal salts which function as Lewis acid (for example ZnCl₂, AlCl₃, TiCl₄ and SnCl₄). The solvent ligands can be, for example, alcohols, amines, acid amides, lactams and sulfones.

Complexes of this type are described in the references mentioned below and the references cited therein:
D. J. Ager, S. A. Laneman, Tetrahedron: Asymmetry, 8, 1997, 3327-3355;
T. Ohkuma, R. Noyori in Comprehensive Asymmetric Catalysis (E. N. Jacobsen, A. Pfaltz, H. Yamamoto, Eds.), Springer, Berlin, 1999, 199-246;
J. M. Brown in Comprehensive Asymmetric Catalysis (E. N. Jacobsen, A. Pfaltz, H. Yamamoto, Eds.), Springer, Berlin, 1999, 122-182;
T. Ohkuma, M. Kitamura, R. Noyori in Catalytic Asymmetric Synthesis, 2^{nd} Edition (I. Ojima, Ed.), Wiley-VCH New York, 2000, 1-110;
N. Zanetti, et al. Organometallics 15, 1996, 860.

The metal complexes of the invention represent homogeneous catalysts or catalyst precursors which can be activated under the reaction conditions and can be used for asymmetric addition reactions on prochiral, unsaturated, organic compounds.

The metal complexes can, for example, be used for asymmetric hydrogenation (addition of hydrogen) of prochiral compounds having carbon-carbon or carbon-heteroatom double bonds. Such hydrogenations using soluble homogeneous metal complexes are described, for example, in Pure and Appl. Chem., Vol. 68, No. 1, pp. 131-138 (1996). Preferred unsaturated compounds to be hydrogenated contain the groups C═C, C═N and/or C═O. According to the invention, metal complexes of ruthenium, rhodium and iridium are preferably used for hydrogenation.

The invention further provides for the use of the metal complexes of the invention as homogeneous catalysts for preparing chiral organic compounds by asymmetric addition of hydrogen onto a carbon-carbon or carbon-heteroatom double bond in prochiral organic compounds.

A further aspect of the invention is a process for preparing chiral organic compounds by asymmetric addition of hydrogen onto a carbon-carbon or carbon-heteroatom double bond in prochiral organic compounds in the presence of a catalyst, which is characterized in that the addition reaction is carried out in the presence of catalytic amounts of at least one metal complex according to the invention.

Preferred prochiral, unsaturated compounds to be hydrogenated can contain one or more, identical or different C═C, C═N and/or C═O groups in open-chain or cyclic organic compounds, with the C═C, C═N and/or C═O groups being able to be part of a ring system or being exocyclic groups. The prochiral unsaturated compounds can be alkenes, cycloalkenes, heterocycloalkenes and also open-chain or cyclic ketones, α,β-diketones, α- or β-ketocarboxylic acids and also their α,β-ketoacetals or -ketals, esters and amides, ketimines and kethydrazones.

Some examples of unsaturated organic compounds are acetophenone, 4-methoxyacetophenone, 4-trifluoromethylacetophenone, 4-nitroacetophenone, 2-chloroacetophenone, corresponding unsubstituted or N-substituted acetophenonebenzylimines, unsubstituted or substituted benzocyclohexanone or benzocyclopentanone and corresponding imines, imines from the group consisting of unsubstituted or substituted tetrahydroquinoline, tetrahyropyridine and dihydropyrrole, and unsaturated carboxylic acids, esters, carboxamides and carboxylic acid salts, for example α- and if desired β-substituted acrylic acids or crotonic acids. Preferred carboxylic acids are acids of the formula
R₀₁—CH═C(R₀₂)—C(O)OH
and also their salts, esters and amides, where R₀₁ is C₁-C₆-alkyl, C₃-C₈-cycloalkyl which may be unsubstituted or bear from 1 to 4 C₁-C₆-alkyl, C₁-C₆-alkoxy, C₁-C₆-alkoxy-C₁-C₄-alkoxy groups as substituents or C₆-C₁₀-aryl, preferably phenyl, which may be unsubstituted or bear from 1 to 4 C₁-C₆-alkyl, C₁-C₆-alkoxy, C₁-C₆-alkoxy-C₁-C₄-alkoxy groups as substituents and R₀₂ is linear or branched C₁-C₆-alkyl (for example isopropyl), cyclopentyl, cyclohexyl, phenyl which may be unsubstituted or substituted as defined above or protected amino (for example acetylamino).

The process of the invention can be carried out at low or elevated temperatures, for example temperatures of from −20 to 150° C., preferably from −10 to 100° C. and particularly preferably from 10 to 80° C. The optical yields are generally better at relatively low temperature than at higher temperatures.

The process of the invention can be carried out at atmospheric pressure or superatmospheric pressure. The pressure can be, for example, from 10⁵ to 2×10⁷ Pa (pascal). Hydrogenations can be carried out at atmospheric pressure or at superatmospheric pressure.

Catalysts are preferably used in amounts of from 0.0001 to 10 mol %, particularly preferably from 0.001 to 10 mol % and in particular from 0.01 to 5 mol %, based on the compound to be hydrogenated.

The preparation of the ligands and catalysts and also the hydrogenation can be carried out without a solvent or in the presence of an inert solvent. In the latter case, one solvent or a mixture of solvents can be used. Suitable solvents are, for example, aliphatic, cycloaliphatic and aromatic hydrocarbons (pentane, hexane, petroleum ether, cyclohexane, methylcyclohexane, benzene, toluene, xylene), aliphatic halogenated hydrocarbons (methylene chloride, chloroform, dichloroethane and tetrachloroethane), nitriles (acetonitrile, propionitrile, benzonitrile), ethers (diethyl ether, dibutyl ether, t-butyl methyl ether, ethylene glycol dimethyl ether, ethylene glycol diethyl ether, diethylene glycol dimethyl ether, tetrahydrofuran, dioxane, diethylene glycol monomethyl or monoethyl ether), ketones (acetone, methyl isobutyl ketone), carboxylic esters and lactones (ethyl acetate or methyl acetate, valerolactone), N-substituted lactams (N-methylpyrrolidone), carboxamides (dimethylamide, dimethylformamide), acyclic ureas (dimethylimidazoline) and sulfoxides and sulfones (dimethyl sulfoxide, dimethyl sulfone, tetramethylene sulfoxide, tetramethylene sulfone) and alcohols (methanol, ethanol, propanol, butanol, ethylene glycol monomethyl ether, ethylene glycol monoethyl ether, diethylene glycol monomethyl ether and water. The solvents can be used either alone or as a mixture of at least two solvents.

The reaction can be carried out in the presence of cocatalysts, for example quaternary ammonium halides (tetrabutylammonium iodide), and/or in the presence of protic acids, for example mineral acids (cf., for example, U.S. Pat. No. 5,371,256, U.S. Pat. No. 5,446,844 and U.S. Pat. No. 5,583,241 and EP-A-0 691 949). The presence of fluorinated alcohols such as 1,1,1-trifluoroethanol can likewise be advantageous for the catalytic reaction.

The metal complexes used as catalysts can be added as separately prepared-isolated compounds or can be formed in situ prior to the reaction and then be mixed with the substrate to be hydrogenated. It can be advantageous to add an additional amount of ligands in the case of the reaction using isolated metal complexes, or in the case of the in-situ preparation, to use an excess of the ligands. The excess can, for example, be from 1 to 6 and preferably from 1 to 2 mol, based on the metal compound used for the preparation.

The process of the invention is generally carried out by placing the catalyst in a reaction vessel and then adding the substrate, if appropriate reaction auxiliaries and the compound to be added on and subsequently starting the reaction. Gaseous compounds to be added on, for example hydrogen or ammonia, are preferably introduced under pressure. The process can be carried out continuously or batchwise in various types of reactor.

The chiral organic compounds which can be prepared according to the invention are active substances or intermediates for the preparation of such substances, in particular in the field of production of flavors and fragrances, pharmaceuticals and agrochemicals.

The following examples illustrate the invention.

A) Preparation of Substituted Ferrocenediphosphines

Abbreviations: Me is methyl, Ph is phenyl, THF is tetrahydrofuran; TBME is tert-butyl methyl ether; nbd=norbornadiene

### EXAMPLE A1

### Methyl Substitution in 3,3′ Positions

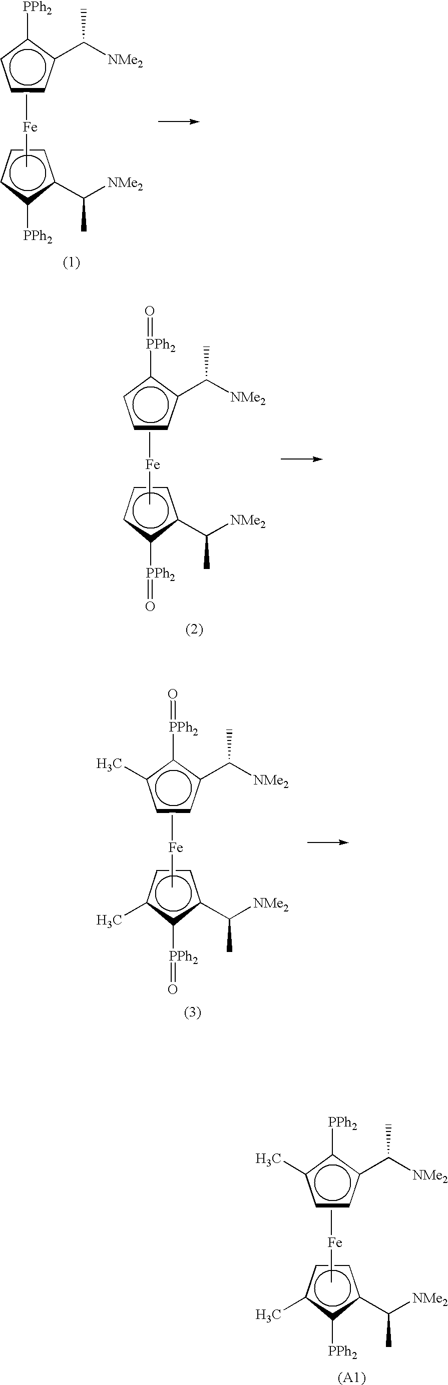 a) Preparation of Compound (2)

2.6 ml (14.4 mmol) of a solution of t-butyl hydroperoxide in nonane (5.5 molar) are added dropwise to a solution of 5 g (7.2 mmol) of the S,R compound (1) in 40 ml of THF at 0° C. while stirring. The cooling is subsequently removed and the mixture is stirred further overnight, resulting in formation of a yellow precipitate. 40 ml of heptane are added, the mixture is filtered, the solid is washed with a little cold diethyl ether and dried under reduced pressure (yield: 88%). The crude product is pure and can be directly used further.

¹H-NMR (CDCl₃), characteristic signals: δ 7.6-7.4 (m, 20H), 5.01 (m, 2H), 4.40 (m, 2H), 4.27 (m, 2H), 3.32 (m, 2H), 1.56 (s, 12H), 1.19 (d, 6H). ³¹P-NMR (CDCl₃): δ +26.3 (s).

b) Preparation of Compound (3)

10.4 ml (16.8 mmol) of n-BuLi (1.6 molar in hexane) are added dropwise to a solution of 4 g (5.6 mmol) of the compound (2) in 200 ml of THF at −78° C. while stirring and the reaction mixture is stirred further at this temperature for 2 hours. 1.05 ml (16.8 mmol) of methyl iodide are then added dropwise at −78° C. and the reaction mixture is stirred further, firstly for 0.5 hour at −78° C., then for 1 hour at −40° C. and finally for 30 minutes at −10° C., before being admixed with 5 ml of water at −10° C. while stirring vigorously. The organic solvent and any unreacted methyl iodide are immediately distilled off under reduced pressure to a maximum of 50° C. and the residue is extracted in methylene chloride/aqueous NaCl solution. The organic phases are collected, dried over sodium sulfate and the solvent is distilled off under reduced pressure on a rotary evaporator. The crude product is obtained as an orange solid which is used further without further purification (yield: >98%).

¹H-NMR (C₆D₆), characteristic signals: δ 7.89-7.7 (m, 8H), 7.1-6.9 (m, 12H), 5.40 (s, 2H), 4.30 (m, 2H), 4.09 (m, 2H), 1.68 (s, 12H), 1.46 (s, 6H), 1.38 (d, 6H). ³¹P-NMR (C₆D₆): δ +27.2 (s).

c) Preparation of Compound (A1):

A suspension of 390 mg (0.53 mmol) of the phosphine oxide (3) and 1.9 ml (10.5 mmol) of HSi(OEt)₃ in 10 ml of toluene is heated to reflux while stirring. 0.19 ml (0.64 mmol) of titanium(IV) isopropoxide is then added dropwise over a period of 20 minutes and the reaction mixture is refluxed overnight. After cooling, the THF is distilled off on a rotary evaporator and the residue is suspended in 2 ml of ethyl acetate and applied to a column. Chromatography (silica gel 60; eluent=ethyl acetate containing 1% of triethylamine) gives the desired product as an orange foam in a yield of 73%.

¹H-NMR (C₆D₆), characteristic signals: δ 7.8-7.7 (m, 4H), 7.4-7.3 (m, 4H), 7.33-7.0 (m, 12H), 4.70 (s, 2H), 4.28 (m, 2H), 3.62 (m, 2H), 1.79 (s, 12H), 1.40 (s, 6H), 1.32 (d, 6H).

³¹P-NMR (C₆D₆): δ −15.3 (s).

### EXAMPLE A2

### Methyl Substitution in the 3 Position

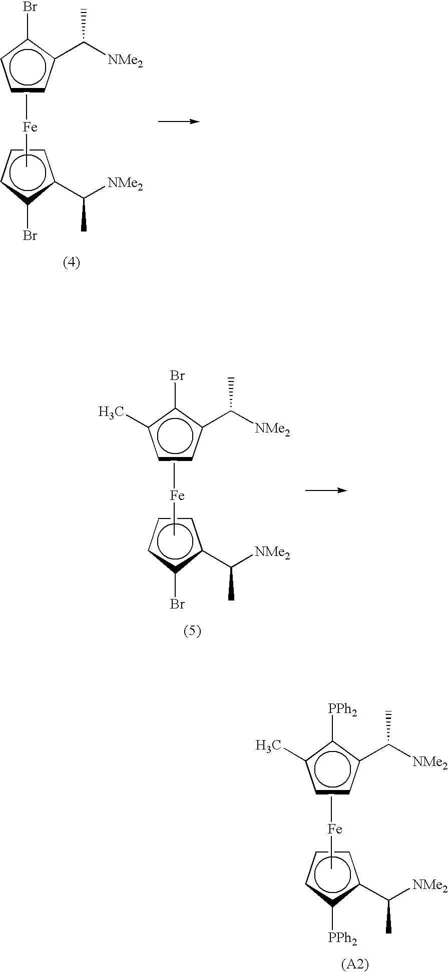 a) Preparation of the Compound (4)

The compound (4) is described in the literature: P. Knochel et al., Tetrahedron: Asymmetry, (1999) 1839-42.

b) Preparation of the Compound (5)

The following solutions are prepared:

Solution a): 2.7 ml (4 mmol) of n-BuLi (1.6 M in hexane) are added dropwise to 0.73 ml (4.1 mmol) of 2,2,6,6-tetramethylpiperidine in 3 ml of THF at 0° C. and the solution is stirred at this temperature for 1 hour.

Solution b): 500 mg (1.03 mmol) of compound (4) in 3 ml of THF.

The solution a) is cooled to −78° C. While stirring, the solution b) is added dropwise over a period of 15 minutes and the reaction mixture is stirred further, firstly for 30 minutes at 78° C., then for 4 hours at −30° C. After cooling back down to −78° C., 0.26 ml (4 mmol) of methyl iodide are added dropwise and the mixture is stirred further for 2 hours at this temperature. The reaction mixture is subsequently admixed with 2 ml of THF/water and extracted with ethyl acetate/water. The organic phases are collected, dried over sodium sulfate and the solvent is distilled off on a rotary evaporator. Chromatographic purification (silica gel 60; eluent=10 dichloromethane/1 methanol containing 1% of triethylamine) gives a mixture of mainly product and starting material. Since this mixture can be separated much more readily after reaction with the phosphine, it is processed further without further purification. A sample is purified by further chromatography for characterization of the product.

¹H-NMR (CDCl₃), characteristic signals: δ 2.10 (s, 6H) 2.08 (s, 6H) 1.95 (s, 3H), 1.45-1.38 (m, 2×3H);

c) Preparation of the Compound (A2):

1.3 ml (2 mmol) of n-BuLi (1.6 molar in hexane) are added dropwise to 340 mg of the product obtained in step b) in 5 ml of diethyl ether at 0° C. and the reaction mixture is stirred at this temperature for 2 hours. After cooling to −78° C., 510 mg of diphenylchlorophosphine are added, the cooling bath is removed and the mixture is stirred overnight. 1 ml of water is added, the mixture is extracted, the organic phase is dried over sodium sulfate and evaporated on a rotary evaporator. Purification by chromatography (silica gel 60; eluent=1 ethyl acetate/5 heptane containing 1% of triethylamine) gives 270 mg of product as an orange solid.

¹H-NMR (C₆D₆), characteristic signals: δ 7.91-7.75 (m, 4H), 7.51-7.37 (m, 4H), 7.23-7.06 (m, 12H) 4.74 (s, 1H), 4.43-4.25 (m, 2H), 4.35 (m, 1H), 4.13 (m, 1H), 4.10 (m, 1H), 3.73 (m, 1H), 1.93 (s, 6H), 1.83 (s, 6H) 1.50-1.43 (m, 6H), 1.18 (d, 3H). ³¹P-NMR (C₆D₆): δ −16.3, −23.2.

### EXAMPLE A3

### Methyl Substitution in the 5 Position

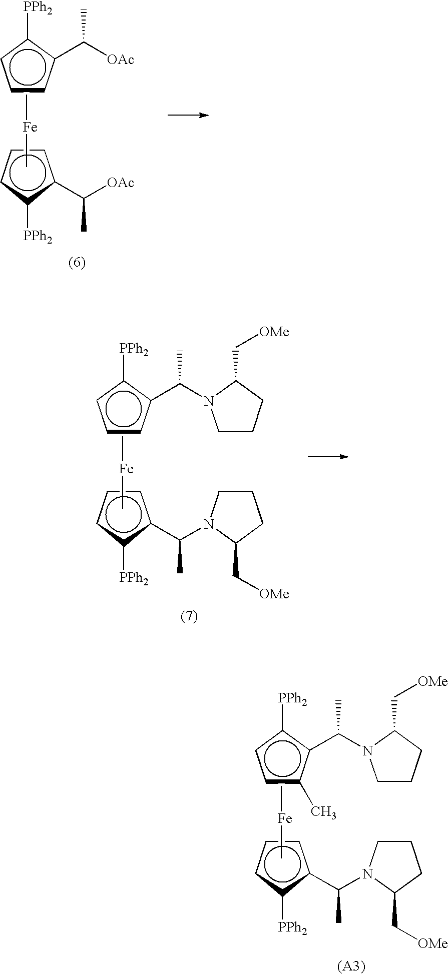 a) Preparation of the Compound (6):

The compound (6) is described in the literature: T. Hayashi et al., J. Organomtal. Chem., 370 (1989) 129-139.

b) Preparation of the Compound (7):

A solution of 5.0 g (6.6 mmol) of the compound (6) and 13.3 g (115 mmol) of O-methyl-(S)-prolinol in 50 ml of acetonitrile and 5 ml of water is stirred at 100° C. for at least 80 hours. After cooling, the solvent and the excess O-methylprolinol are distilled off under reduced pressure on a rotary evaporator. The residue is taken up in 20 ml of TBME and washed a number of times with water. The organic phase is dried over sodium sulfate and evaporated on a rotary evaporator. The crude product is purified by chromatography (silica gel 60; eluent=1 ethyl acetate/4 heptane and 1% of triethylamine). The product is obtained as an orange, solid foam (yield: 93%).

¹H-NMR (C₆D₆), characteristic signals: δ 7.52-7.45 (m, 4H), 7.41-7.34 (m, 8H), 7.01-6.9 (m, 12H) 4.53 (m, 2H), 4.54-4.47 (m, 2H), 4.20 (m, 2H), 3.17 (m, 2H), 3.11 (s, 6H), 1.52 (d, 6H). ³¹P-NMR (C₆D₆): δ −b 24.2.

c) Preparation of the Compound (A3):

4 ml (5.2 mmol) of s-butyllithium (s-BuLi) (1.3 M in cyclohexane) are added dropwise to a solution of 2 g (2.4 mmol) of the compound (7) in 35 ml of TBME at 0° C. and the reaction mixture is stirred further at 0° C. for 2-hours. It is then cooled to −78° C. and 0.195 ml (3.1 mmol) of methyl iodide is added. After one hour, the cooling bath is removed. The temperature is allowed to rise to 0° C. and the mixture is stirred for another 2 hours at 0° C. The reaction mixture is poured into ice water, the organic phase is dried over sodium sulfate and evaporated under reduced pressure on a rotary evaporator. The residue is purified by chromatography (silica gel 60; eluent—1 ethyl acetate/4 heptane and 1% of triethylamine). The product is obtained as a yellow solid in a yield of 71%.

¹H-NMR (C₆D₆), characteristic signals: δ 7.50-7.46 (m, 4H), 7.44-7.33 (m, 8H), 7.01-6.93 (m, 12H) 4.60 (m, 1H), 4.59-4.49 (m, 1H), 4.42 (m, 1H), 4.39 (m, 1H), 4.35-4.27 (m, 2H), 3.29 (m, 1H), 3.12 (s, 3H), 3.09 (s, 3H), 2.14 (s, 3H), 1.70 (d, 3H), 1.42 (d, 3H).

³¹P-NMR (C₆D₆): δ −23.3; −24.1.

### EXAMPLE A4

### Bromine Substitution in the 5,5′ Positions

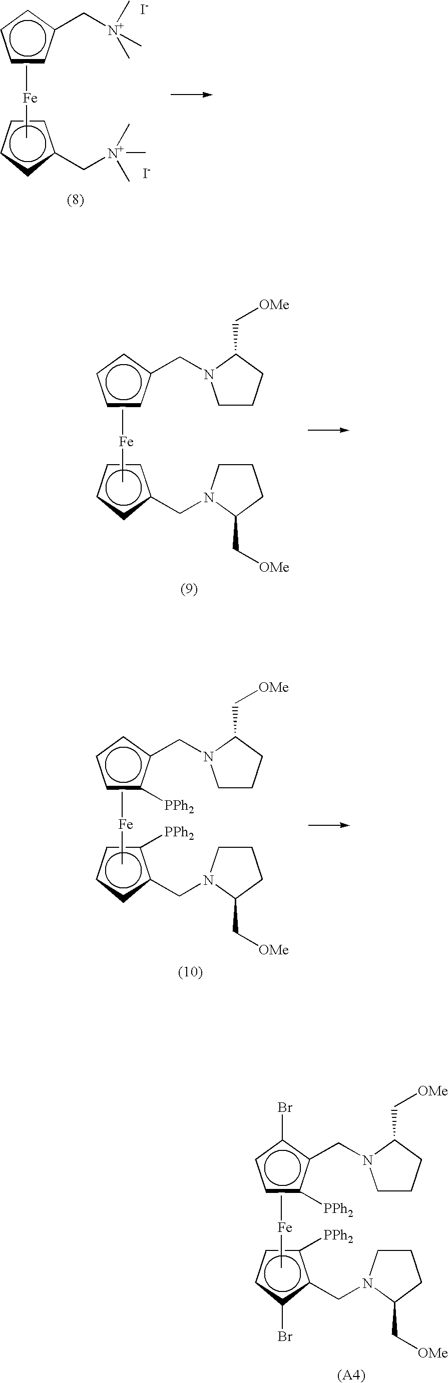 a) Preparation of Compound (8)

Compound (8) is prepared as described by C. Glidewell et al. in J. Organometal. Chem. 527 (1997), pages 259-261.

b) Preparation of Compound (9)

4.94 g (42.88 mmol) of (S)-2-(methoxymethyl)pyrrolidine are added to 5.01 g (8.57 mmol) of the compound (8) in 600 ml of dry acetonitrile and the reaction mixture is stirred at 100° C. for 72 hours. After cooling, the solvent is distilled off on a rotary evaporator. The residue is extracted in saturated aqueous NaHCO₃/methylene chloride, the organic phases are dried over sodium sulfate and evaporated on a rotary evaporator. Chromatography (silica gel 60; eluent=1 THF/2 heptane and 2% of triethylamine) gives the desired product as an orange oil.

¹H-NMR (C₆D₆), characteristic signals: δ 4.16 (m, 2H), 4.11 (m, 2H), 3.98 (m, 4H), 3.95-3.90 (d, 2H), 3.50-3.40 (m, 4H), 3.24-3.19 (m, 2H), 3.20 (s, 6H), 2.97 (m, 2H), 2.79 (m, 2H), 2.21 (m, 2H), 1.81-1.42 (m, 8H).

c) Preparation of Compound (10)

730 mg (1.66 mmol) of the compound (9) are dissolved in 2 ml of TBME. While stirring, 3.18 ml (4.14 mmol) of s-BuLi (1.3 molar solution in cyclohexane) are slowly added dropwise at −78° C. The reaction mixture is stirred for 1 hour at −78° C. and then for 4 hours at −30° C. It is then cooled back down to −78° C. and 988 mg (4.48 mmol) of diphenylchlorophosphine are added. After 15 minutes, the cooling is removed and the reaction mixture is stirred further overnight. It is then extracted with water/TBME, the organic phase is dried over sodium sulfate and the solvent is distilled off under reduced pressure on a rotary evaporator. Chromatography (silica gel 60; eluent firstly methylene chloride until Cl—P Ph₂ has been eluted, then 1 THF/5 heptane and 1% of triethylamine) gives the desired product as a yellow solid (yield: 70%).

¹H-NMR (C₆D₆), some characteristic signals: δ 7.53 (m, 4H), 7.29 (m, 4H), 7.05-6.96 (m, 12H), 4.64-4.59 (m, 2H), 4.39 (m, 2H), 4.17 (m, 2H), 3.63 (m, 2H), 3.37 (m, 2H), 3.21 (s, 6H). ³¹P-NMR (C₆D₆): δ −22.6 (s).

d) Preparation of Compound (A4)

A solution of 510 mg (0.63 mmol) of the compound (10) in 10 ml of TBME is cooled to −78° C. and 1.05 ml (1.57 mmol) of t-butyllithium (1.5 molar solution in pentane) is slowly added dropwise. The temperature is allowed to rise to −30° C. and the mixture is stirred further, firstly for 2 hours at this temperature and subsequently for 30 minutes at 0° C. After cooling back down to −78° C., a solution of 408 mg (1.57 mmol) of 1,2-dibromotetrafluoroethane in 1 ml of THF is slowly added dropwise and the mixture is stirred further for 30 minutes at −78° C. The cooling bath is then removed and the mixture is stirred for another 1 hour. The reaction mixture is admixed with 2 ml of water and extracted with methylene chloride. The organic phases are dried over sodium sulfate and evaporated on a rotary evaporator. Purification by chromatography (silica gel 60; eluent=20 heptane/1 ethyl acetate and 1% of triethylamine) gives the product as a yellow solid.

¹H-NMR (C₆D₆), some characteristic signals: δ 7.35-7.29 (m, 4H), 7.23-7.16 (m, 4H), 7.02-6.98 (m, 6H), 6.88-6.79 (m, 6H), 4.77 (m, 2H), 4.67 (m, 2H), 3.88 (m, 2H), 3.52 (m, 2H), 3.28 (s, 6H), 3.08 (m, 2H). ³¹P-NMR (C₆D₆): δ −23.1 (s).

### EXAMPLE A5

### Trimethylsilyl Substitution in the 5 Position

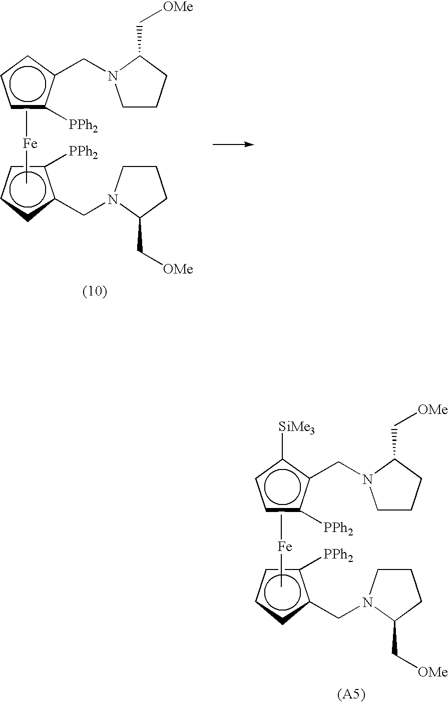

A solution of 500 mg (0.62 mmol) of the compound (10) in 20 ml of TBME is cooled to −78° C. and 0.5 ml (0.75 mmol) of t-BuLi (1.5 molar solution in pentane) is slowly added dropwise. The mixture is subsequently stirred for 2 hours at a temperature in the range from −30° C. to −15° C. After cooling back down to −78° C., 0.1 ml (0.8 mmol) of trimethylchlorosilane is added and the mixture is stirred further for 30 minutes at −78° C. The cooling bath is then removed and the mixture is stirred for another 1 hour. The reaction mixture is admixed with 2 ml of water and then extracted with water. The organic phase is dried over sodium sulfate and evaporated on a rotary evaporator. Purification by chromatography (silica gel 60; eluent=20 heptane/1 ethyl acetate and 1% of triethylamine) gives the product as an orange solid.

¹H-NMR (C₆D₆), some characteristic signals: δ 7.51-7.45 (m, 4H), 7.31-7.22 (m, 4H), 7.02-6.88 (m, 12H), 4.77 (m, 1H), 4.73-4.65 (m, 2H), 4.12 (m, 1H), 3.98 (m, 1H), 3.54 (m, 1H), 3.34 (s, 3H), 3.22 (m, 3H), 0.57 (m, 9H). ³¹P-NMR (C₆D₆): δ −23.3, −23.5.

### EXAMPLE A6

### Trimethylsilyl Substitution in the 5,5′ Positions

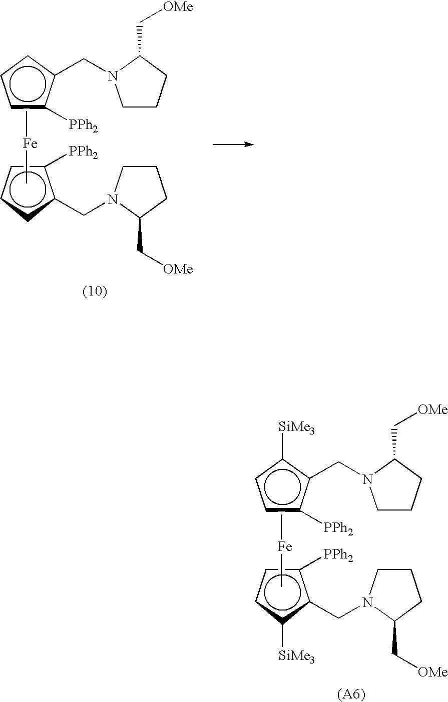

A solution of 500 mg (0.62 mmol) of the compound (10) in 20 ml of TBME is cooled to −78° C. and 0.9 ml (1.4 mmol) of t-BuLi (1.5 molar solution in pentane) is slowly added dropwise. The mixture is subsequently stirred for 2 hours at a temperature in the range from −30° C. to −15° C. After cooling back down to −78° C., 0.2 ml (1.6 mmol) of trimethylchlorosilane is added and the mixture is stirred further for 30 minutes at −78° C. The cooling bath is then removed and the mixture is stirred for another 1 hour. The reaction mixture is admixed with 2 ml of water and then extracted with water. The organic phase is dried over sodium sulfate and evaporated on a rotary evaporator. Purification by chromatography (silica gel 60; eluent 20 heptane/1 ethyl acetate and 1% of triethylamine) gives the product as an orange solid which, according to 1H- and 31P-NMR, is a mixture of two atropic isomers.

¹H-NMR (C₆D₆), some characteristic signals:

Signals of the O—CH₃ groups: δ 3.36 and 3.32 (two s with integral ratio ˜33:67, total 6H).

Signals of the Si(CH₃) group: δ 0.65 and 0.03 (two s with integral ratio ˜67:33, total 18H).

³¹P-NMR (C₆D₆): δ −24.5 (s, large signal), −28.1 (s, smaller signal).

### EXAMPLE (A7)

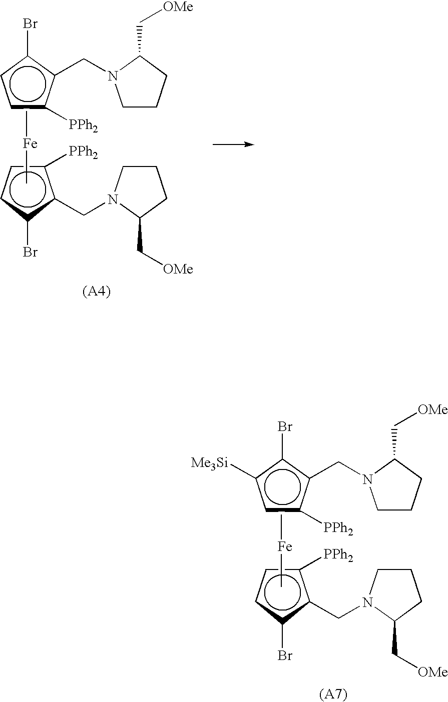

The following solutions are prepared:

Solution a): 0.86 ml (1.37 mmol) of n-butyllithium (1.6 M in hexane) is added dropwise to 0.232 ml (1.37 mmol) of 2,2,6,6-tetramethylpiperidine in 1 ml of THF at 0° C. and the solution is stirred at this temperature for 1 hour.

Solution b): 220 mg (0.23 mmol) of the compound (A4) in 10 ml of THF.

The solution a) is cooled to −78° C. While stirring, the solution b) is added dropwise over a period of 15 minutes and the reaction mixture is stirred further, firstly for 30 minutes at −78° C., then for 4 hours at from −15° to −20° C. After cooling back down to −78° C., 0.173 ml (1.37 mmol) of trimethylchlorosilane is added dropwise and the mixture is then stirred further for 2 hours at −20° C. The reaction mixture is cooled back down to −78° C. and stirred overnight at this temperature. The temperature is then allowed to rise slowly to +10° C. The reaction mixture is subsequently admixed with a little water and extracted with ethyl acetate/water. The organic phases are collected, dried over sodium sulfate and the solvent is distilled off on a rotary evaporator. After purification by chromatography (silica gel 60; eluent=1 ethyl acetate/15 heptane containing 1% of triethylamine), an orange product is isolated.

¹H-NMR (CDCl₃), some characteristic signals: δ 7.79-7.73 (m, 2H), 7.65-7.59 (m, 2H), 7.04-6.94 (m, 6H), 3.47 (s, 3H), 3.44 (s, 3H), 0.52 (s, 9H). ³¹P-NMR (C₆D₆): δ −24.6; −24.9.

B) Preparation of Metal Complexes

### EXAMPLE B1

5.1 mg (0.0136 mmol) of [Rh(nbd)₂]BF₄ and 10.4 mg (0.0163 mmol) of the diphosphine from Example A6 are weighed into a Schlenk vessel provided with a magnetic stirrer and the air is displaced by means of vacuum and argon. Addition of 0.8 ml of degassed methanol with stirring gives an orange solution of the metal complex (catalyst solution). A uniform, C₂-symmetric complex is formed.

¹H-NMR (CDCl₃), some characteristic signals: Signals of the O—CH₃ groups: δ 3.14 (s, 6H); signals of the Si(CH₃)₃ group: δ 0.21 (s, 18H); ³¹P-NMR (CDCl₃): δ +25.6 (d, J_{Rh—P}=170 Hz).

C) Use Examples

### EXAMPLE C1

### Hydrogenation of Unsaturated Compounds

The method of carrying out the hydrogenations and the determination of the optical yields ee is described in general terms by W. Weissensteiner et al. in Organometallics 21 (2002), pages 1766-1774. The catalysts are in each case prepared “in situ” by mixing of ligand and metal complex as catalyst precursor (unless indicated otherwise=[Rh(norbornadiene)₂]BF₄) in the solvent. Unless indicated otherwise, the substrate concentration is 0.25 mol/l, the molar ratio of substrate to metal=200 and the molar ratio of ligand to metal=1.05.

Hydrogenations:

Reaction conditions for the substrates MAC, DMI, MCA, EAC:

Catalyst precursor=[Rh(norbornadiene)₂]BF₄; solvent=MeOH; hydrogen pressure=1 bar; temperature=25° C.; reaction time 1 hour.

The hydrogenations of EAC are carried out in ethanol in the presence of 5% (v/v) of CF₃CH₂OH. In the case of EAC, the ee is determined by means of gas chromatography using a chiral column [Lipodex E (30 m); 130° C. isothermal; 190 KPa of H₂].

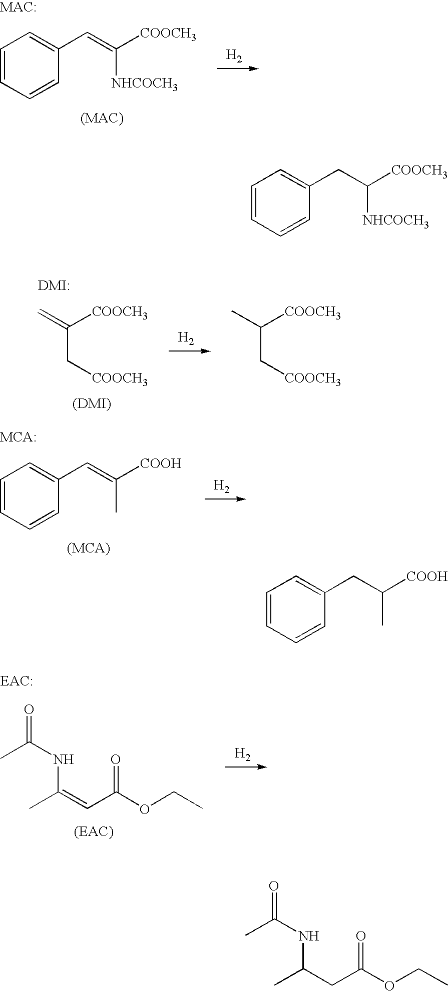

Reaction conditions for the substrate MPG:

Catalyst precursor=[Rh(norbornadiene)Cl]₂]; solvent=toluene; hydrogen pressure=80 bar (8×10⁶ Pa); temperature=25° C.; reaction time=16 hours.

MPG:

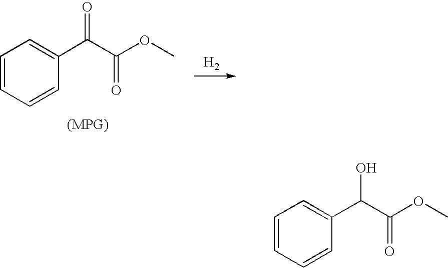

Reaction conditions for the substrate EOV:

Catalyst precursor=[RuI₂(p-cumene)]₂; solvent=ethanol; addition: 0.06 ml of 1N HCl per 10 ml of ethanol; hydrogen pressure=80 bar; temperature=80° C.; reaction time=16 hours.

The determination of the ee is carried out by means of gas chromatography using a chiral column [Lipodex E (30 m)] after derivatization using trifluoroacetic anhydride.

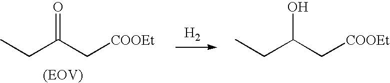

Reaction conditions for the substrate MEA:

Molar ratio of substrate to metal=100; catalyst precursor=[Ir(COD)Cl]₂; solvent=toluene; additions: 2 equivalents of tetrabutylammonium iodide per equivalent of Ir and 0.03 ml of trifluoroacetic acid per 10 ml of toluene; hydrogen pressure=80 bar; temperature=25° C.; reaction time=16 hours.
MEA:

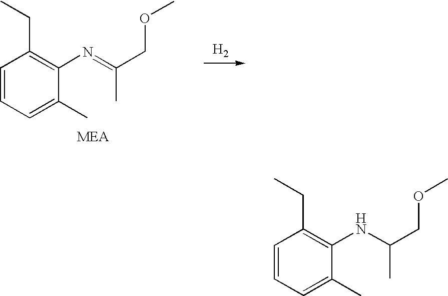

The results of the hydrogenation are reported in Tables 1 and 2 below. “ee” is the enantiomeric excess. The configuration is indicated in parentheses. It can be seen from the results using the comparative ligand and substituted ligands in Table 1 that the substitution can surprisingly influence and invert the configuration. Furthermore, the increase in the optical yields on introduction of substituents can be seen.

TABLE 1

| Substrate | 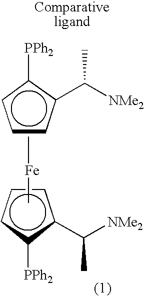 | 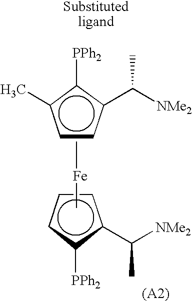 | 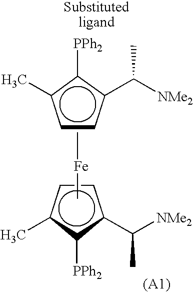 |
|---|---|---|---|
| DMI | 15% ee (R) | | 43% ee (S) |
| EAC | 5% ee (S) | 13% ee (R) | 47% ee (R) |
| EOV | 23% ee (S) | | 40% ee (S) |

TABLE 2
Hydrogenations using ligands according to the invention

| Substrate | 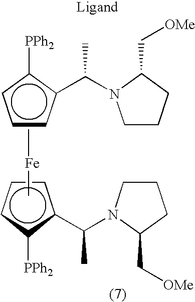 | 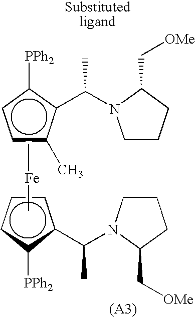 | Substituted ligand | Substituted ligand |
|---|---|---|---|---|
| MAC | 89% ee (S) | 92% ee (S) | | |
| DMI | 31% ee (S) | 99% ee (S) | | |
| EOV | 50% ee (S) | 65% ee (S) | | |
| MEA | 46% ee (S) | 60% ee (S) | | |

| Substrate | 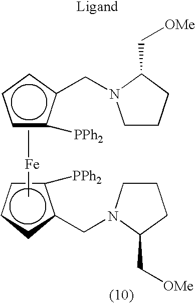 | 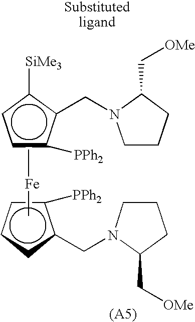 | 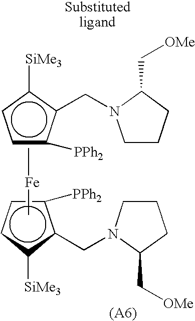 | 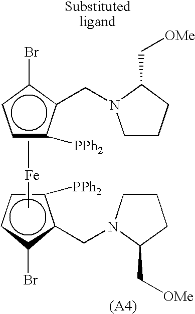 |
|---|---|---|---|---|
| MAC | 78% ee (R) | | 94% ee (R) | |
| MCA | 64% ee (S) | | | 84% ee (S) |
| MPG | 14% ee (R) | 22% ee (S) | 35% ee (R) | 24% ee (R) |
| MEA | 21% ee (R) | | | 29% ee (R) |

1. A compound of the formula I or I′ in the form of a racemate, a mixture of stereoisomers or an optically pure stereoisomer, 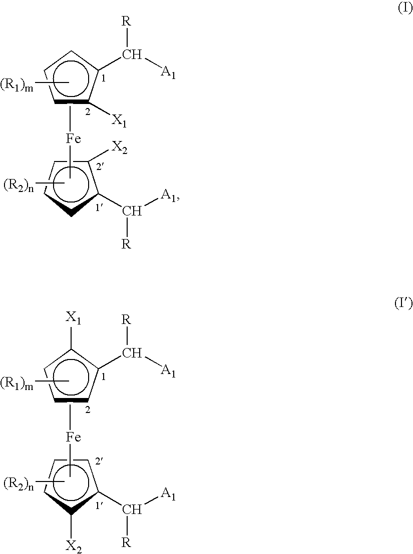
where
R is hydrogen or unsubstituted or F—, Cl—, OH—, C₁-C₄-alkyl- or C₁-C₄-alkoxy-substituted C₁-C₈-alkyl, C₃-C₈-cycloalkyl, C₆-C₁₀-aryl or C₇-C₁₁-aralkyl;
X₁ and X₂ are each, independently of one another, a secondary phosphino group;
A₁ is an amino group; or
A₁ is an —OR₃ radical, where R₃ is hydrogen or unsubstituted or F—, C₁-C₄-alkyl-, C₁-C₄-alkoxy-, phenyl- or N(C₁-C₄-alkyl)₂-substituted C₁-C₁₈-alkyl, C₃-C₈-cycloalkyl, C₆-C₁₀-aryl, C₇-C₁₁-aralkyl or C₁-C₁₈-acyl;
R₁ and R₂ are each, independently of one another, a halogen atom or a substituent bound to the cyclopentadienyl rings via a C atom, N atom, S atom, Si atom, a P(O) group or P(S) group;
m is from 1 to 3, and
n is 0 or from 1 to 3.

2. The compound as claimed in claim 1, characterized in that R is hydrogen, methyl, ethyl, cyclohexyl, benzyl or phenyl.

3. The compound as claimed in claim 1, characterized in that the secondary phosphino groups X₁ and X₂ contain two identical or two different hydrocarbon radicals and in that the secondary phosphino groups X₁ and X₂ are identical or different.

4. The compound as claimed in claim 1, characterized in that the radicals X₁ and X₂ are identical or different acyclic sec-phosphino groups in each case selected from the group consisting of —P(C₁-C₆-alkyl)₂, —P(C₅-C₈-cycloalkyl)₂, —P(C₇-C₁₂-bicycloalkyl)₂, —P(o-furyl)₂, —P(C₆H₅)₂, —P[2-(C₁-C₆-alkyl)C₆H₄]₂, —P[3-(C₁-C₆-alkyl)C₆H₄]₂, —P[4-(C₁-C₆-alkyl)C₆H₄]₂, —P[2-(C₁-C₆-alkoxy)C₆H₄]₂, —P[3-(C₁-C₆-alkoxy)C₆H₄]₂, —P[4-(C₁-C₆alkoxy)C₆H₄]₂, —P[2-(trifluoromethyl)C₆H₄]₂, —P[3-(trifluoromethyl)C₆H₄]₂, —P[4-(trifluoromethyl)C₆H₄]₂, —P[3,5-bis(trifluoromethyl)C₆H₃]₂, —P[3,5-bis(C₁-C₆-alkyl)₂C₆H₃]₂, —P[3,5-bis(C₁-C₆-alkoxy)₂C₆H₃]₂, —P[3,4,5-tris(C₁-C₆-alkoxy)₂C₆H₃]₂ and —P[3,5-bis(C₁-C₆-alkyl)₂₋₄-(C₁-C₆-alkoxy)C₆H₂]₂, or cyclic phosphino groups selected from the group consisting of 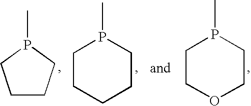
which are unsubstituted or monosubstituted or multiply substituted by C₁-C₄-alkyl, C₁-C₄-alkoxy, C₁-C₄-alkoxy-C₁-C₂-alkyl, phenyl, benzyl, benzyloxy, C₁-C₄-alkylidenedioxyl or unsubstituted or phenyl-substituted methylenedioxyl.

5. The compound as claimed in claim 1, characterized in that X₁ and X₂ are each —P(CH₃)₂, —P(i-C₃H₇)₂, —P(n-C₄H₉)₂, —P(i-C₄H₉)₂, —P(C₆H₁₁)₂, —P(norbornyl)₂, —P(o-furyl)₂, —P(C₆H₅)₂,
P[2-(methyl)C₆H₄]₂, P[3-(methyl)C₆H₄]₂, —P[4-(methyl)C₆H₄]₂, —P[2-(methoxy)C₆H₄]₂, —P[3-(methoxy)C₆H₄]₂, —P[4-(methoxy)C₆H₄]₂, —P[3-(trifluoromethyl)C₆H₄]₂, —P[4-(trifluoromethyl)C₆H₄]₂, —P[3,5-bis(trifluoromethyl)C₆H₃]₂, —P[3,5-bis(methyl)C₆H₃]₂, —P[3,5-bis(methoxy)C₆H₃]₂, —P[3,4,5-tri(methoxy)C₆H₂]₂ and —P[3,5-bis(methyl)₂₋₄-(methoxy)C₆H₂]₂ or a group having one of the formulae 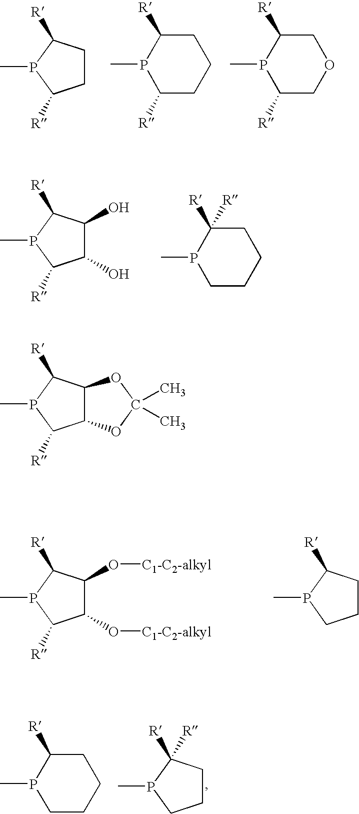
where
R′ is methyl, ethyl, methoxy, ethoxy, phenoxy, benzyloxy, methoxymethyl, ethoxymethyl or benzyloxymethyl and R″ has the same meanings as R′.

6. The compound as claimed in claim 1, characterized in that A₁ is —NH₂, —NHR₅ or —NR₅R₆, where R₅ and R₆ are each, independently of one another, a substituted or unsubstituted aliphatic, cycloaliphatic or aromatic hydrocarbon radical or R₅ and R₆ together with the N atom form an N-heterocyclic ring which may contain further heteroatoms from the group consisting of O, S or N(C₁-C₄-alkyl).

7. The compound as claimed in claim 6, characterized in that R₅ and R₆ are each methyl, ethyl, the isomers of propyl and butyl, phenyl, benzyl, cyclohexyl or R₅ and R together are tetramethylene, pentamethylene or 3-oxapentylene, which may be unsubstituted or substituted by C₁-C₄-alkyl, C₅-C₆-cycloalkyl, phenyl, benzyl, C₁-C₄-alkoxy, C₁-C₄-alkoxymethyl, C₁-C₄-alkoxyethyl, (C₁-C₄-alkyl)₂N—, (C₁-C₄-alkyl)₂N-methyl and (C₁-C₄-alkyl)₂N-ethyl.

8. The compound as claimed in claim 1, characterized in that the substituents R₁ and R₂ are present once (m is 1 and n is 0), each present once (m and n are each 1), present twice (either m or n is 2) or present three times (m is 2 and n is 1) on the cyclopentadienyl ring or rings.

9. The compound as claimed in claim 1, characterized in that the positions of the substituents R₁ and R₂ are the 3, 3′, 5 and 5′ positions and preferred substitution patterns are the 3 position, the 3 and 3′ positions, the 5 position and the 5 and 5′ positions.

10. The compound as claimed in claim 1, characterized in that the substituents R₁ and R₂ are selected from among C₁-C₄-alkyl, substituted or unsubstituted phenyl, tri(C₁-C₄-alkyl)Si, triphenylsilyl, halogen, —SR₀₆, —CH₂OH, —CHR₀₆OH, —CR₀₆R′₀₆OH, —CH₂O—R₀₆, —CH(O), —CO₂H, —CO₂R₀₆ and —P(O)(R₀₃)₂, where R₀₆ is a hydrocarbon radical having from 1 to 10 carbon atoms and R′₀₆O independently has one of the meanings of R′₀₆, and, where R₀₃ is hydrogen, C₁-C₈-alkyl, C₅-C₆-cycloalkyl, phenyl or benzyl.

11. A compound of the formula IV 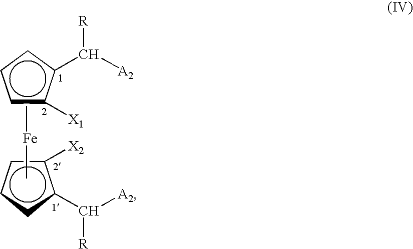
where R is hydrogen or unsubstituted or F—, Cl—, OH—, C₁-C₄-alkyl- or C₁-C₄-alkoxy-substituted C₁-C₈-alkyl, C₃-C₈-cycloalkyl, C₆-C₁₀-aryl or C₇-C₁-aralkyl; A₂ is open-chain or cyclic sec-amino having at least one asymmetric carbon atom, and X₁ and X₂ are each, independently of one another, a secondary phosphino group.

12. A complex of a metal selected from the group of TM8 metals, preferably Cu, Ag, Au, Ni, Co, Rh, Pd, Ir, Ru and Pt, with one of the compounds of the formula I or I′ as claimed in claim 1, as ligand.

13. The metal complex as claimed in claim 12 which corresponds to one of the formulae XII and XIII,
A₃MeLᵣ (XII),
(A₃MeLᵣ)^{(z+)}(E⁻)_{z} (XIII),
where A₃ is one of the compounds of the formula I or I′ or IV,
L represents identical or different monodentate, anionic or nonionic ligands or L represents identical or different bidentate, anionic or nonionic ligands;
r is 2, 3 or 4 when L is a monodentate ligand or n is 1 or 2 when L is a bidentate ligand;
z is 1, 2 or 3;
Me is a metal selected from the group consisting of Rh, Ir and Ru; with the metal having the oxidation states 0, 1, 2, 3 or 4;
E⁻ is the anion of an oxo acid or complex acid; and
the anionic ligands balance the charge of the oxidation states 1, 2, 3 or 4 of the metal.

14. The metal complex as claimed in claim 12 which corresponds to the formula XVI,
[RuₐH_{b}Z_{c}(A₃)_{d}Lₑ]_{f}(E^{k})_{g}(S)ₕ (XVI),
where
Z is Cl, Br or I; A₃ is a compound of the formula I or I′ or IV; L represents identical or different ligands; E⁻ is the anion of an oxo acid, mineral acid or complex acid; S is a solvent capable of coordination as ligand; and a is from 1 to 3, b is from 0 to 4, c is from 0 to 6, d is from 1 to 3, e is from 0 to 4, f is from 1 to 3, g is from 1 to 4, h is from 0 to 6 and k is from 1 to 4, with the overall complex being uncharged.

15. The use of the metal complexes as claimed in claim 12 as homogeneous catalysts for preparing chiral organic compounds, preferably for the asymmetric addition of hydrogen onto a carbon-carbon or carbon-heteroatom double bond in prochiral organic compounds.

16. A process for preparing chiral organic compounds by asymmetric addition of hydrogen onto a carbon-carbon or carbon-heteroatom double bond in prochiral organic compounds in the presence of a catalyst, characterized in that the addition reaction is carried out in the presence of catalytic amounts of at least one metal complex as claimed in claim 12.

17. A complex of a metal selected from the group of TM8 metals, preferably Cu, Ag, Au, Ni, Co, Rh, Pd, Ir, Ru and Pt, with one of the compounds of the formula IV as claimed in claim 11, as ligand.
